SEQUENCE LISTING (28 sequences)

SEQ ID NO: 1 - protein, 967 aa, synthetic construct
  REGION 1..967: note = Synthetic: Cas13d (RfxCas13d)
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 2 - protein, 967 aa, synthetic construct
  REGION 1..967: note = Synthetic: dCas13d (dRfxCas13d)
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 3 - protein, 919 aa, synthetic construct
  REGION 1..919: note = Synthetic: UrCas13d
MAKKNKMKPRELREAQKKARQLKAAEINNNAAPAIAAMPAAEVIAPAAEKKKSSVKAAGMKSILVSENKMYITSFGKGNSAVLEYEVDNNDYNQTQLSSKDNSNIQLGGVNEVNITFSSKHGFESGVEINTSNPTHRSGESSPVRGDMLGLKSELEKRFFGKTFDDNIHIQLIYNILDIEKILAVYVTNIVYALNNMLGVKGSESHDDFIGYLSTNNIYDVFIDPDNSSLSDDKKANVRKSLSKFNALLKTKRLGYFGLEEPKTKDNRVSQAYKKRVYHMLAIVGQIAQCVFADKSGAKRFDLYSFINNIDPEYRDTLDYLVEERLKSINKDFIEDNKVNISLLIDMMKGYEADDIIRLYYDFIVLKSQKNLGFSIKKLREKMLDEYGFRFKDKQYDSVRSKMYKLMDFLLFCNYYRNDIAAGESLVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDKITDDRISGILKLKEKGKGIHGLRNFITNNVIESSRFVYLIKYANAQKIREVAKNEKVVMFVLGGIPDTQIERYYKSCVEFPDMNSSLGVKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDKSPNLFLKKNERLRKCVEVDINNADSSMTRKYANCIAALTVVRELKEYIGDICTVDSYFSIYHYVMQRCITKRENDTKQEEKIKYEDDLLKNHGYTKDFVKALNSPFGYNIPRFKNLSIEQLFDRNEYLTEK

SEQ ID NO: 4 - protein, 933 aa, synthetic construct
  REGION 1..933: note = Synthetic: RffCas13d
MKKKMSLREKREAEKQAKKAAYSAASKNTDSKPAEKKAETPKPAEIISDNSRNKTAVKAAGLKSTIISGDKLYMTSFGKGNAAVIEQKIDINDYSFSAMKDTPSLEVDKAESKEISFSSHHPFVKNDKLTTYNPLYGGKDNPEKPVGRDMLGLKDKLEERYFGCTFNDNLHIQIIYNILDIEKILAVHSANITTALDHMVDEDDEKYLNSDYIGYMNTINTYDVFMDPSKNSSLSPKDRKNIDNSRAKFEKLLSTKRLGYFGFDYDANGKDKKKNEEIKKRLYHLTAFAGQLRQWSFHSAGNYPRTWLYKLDSLDKEYLDTLDHYFDKRFNDINDDFVTKNATNLYILKEVFPEANFKDIADLYYDFIVIKSHKNMGFSIKKLREKMLECDGADRIKEQDMDSVRSKLYKLIDFCIFKYYHEFPELSEKNVDILRAAVSDTKKDNLYSDEAARLWSIFKEKFLGFCDKIVVWVTGEHEKDITSVIDKDAYRNRSNVSYFSKLMYAMCFFLDGKEINDLLTTLINKFDNIANQIKTAKELGINTAFVKNYDFFNHSEKYVDELNIVKNIARMKKPSSNAKKAMYHDALTILGIPEDMDEKALDEELDLILEKKTDPVTGKPLKGKNPLRNFIANNVIENSRFIYLIKFCNPENVRKIVNNTKVTEFVLKRIPDAQIERYYKSCTDSEMNPPTEKKITELAGKLKDMNFGNFRNVRQSAKENMEKERFKAVIGLYLTVVYRVVKNLVDVNSRYIMAFHSLERDSQLYNVSVDNDYLALTDTLVKEGDNSRSRYLAGNKRLRDCVKQDIDNAKKWFVSDKYNSITKYRNNVAHLTAVRNCAEFIGDITKIDSYFALYHYLIQRQLAKGLDHERSGFDRNYPQYAPLFKWHTYVKDVVKALNAPFGYNIPRFKNLSIDALFDRNEIKKNDGEKKSDD

SEQ ID NO: 5 - protein, 944 aa, synthetic construct
  REGION 1..944: note = Synthetic: RaCas13d
MAKKSKGMSLREKRELEKQKRIQKAAVNSVNDTPEKTEEANVVSVNVRTSAENKHSKKSAAKALGLKSGLVIGDELYLTSFGRGNEAKLEKKISGDTVEKLGIGAFEVAERDESTLTLESGRIKDKTARPKDPRHITVDTQGKFKEDMLGIRSVLEKKIFGKTFDDNIHVQLAYNILDVEKIMAQYVSDIVYMLHNTDKTERNDNLMGYMSIRNTYKTFCDTSNLPDDTKQKVENQKREFDKIIKSGRLGYFGEAFMVNSGNSTKLRPEKEIYHIFALMASLRQSYFHGYVKDTDYQGTTWAYTLEDKLKGPSHEFRETIDKIFDEGFSKISKDFGKMNKVNLQILEQMIGELYGSIERQNLTCDYYDFIQLKKHKYLGFSIKRLRETMLETTPAECYKAECYNSERQKLYKLIDFLIYDLYYNRKPARIEEIVDKLRESVNDEEKESIYSVEAKYVYESLSKVLDKSLKNSVSGETIKDLQKRYDDETANRIWDISQHSISGNVNCFCKLIYIMTLMLDGKEINDLLTTLVNKFDNIASFIDVMDELGLEHSFTDNYKMFADSKAICLDLQFINSFARMSKIDDEKSKRQLFRDALVILDIGNKDETWINNYLDSDIFKLDKEGNKLKGARHDFRNFIANNVIKSSRFKYLVKYSSADGMIKLKTNEKLIGFVLDKLPETQIDRYYESCGLDNAVVDKKVRIEKLSGLIRDMKFDDFSGVKTSNKAGDNDKQDKAKYQAIISLYLMVLYQIVKNMIYVNSRYVIAFHCLERDFGMYGKDFGKYYQGCRKLTDHFIEEKYMKEGKLGCNKKVGRYLKNNISCCTDGLINTYRNQVDHFAVVRKIGNYAAYIKSIGSWFELYHYVIQRIVFDEYRFALNNTESNYKNSIIKHHTYCKDMVKALNTPFGYDLPRYKNLSIGDLFDRNNYLNKTKESIDANSSIDSQ

SEQ ID NO: 6 - protein, 946 aa, synthetic construct
  REGION 1..946: note = Synthetic: AdmCas13d
MNNKRKTKAKAAGLKSVFFDQKQAVLTTFAKGNNSQIEKKVVNSEVKDLRQPPAFDLELKEKTFYISGKNNINTSRENPLASASLPLSKRQRIRAERIKRAREENRPYHNVKRVGEDDLRAKADLEKHYFGKEYSDNLKIQIIYNILDINKIISPYINDIVYSMNNLARNDEYIDGKIDVIGSLSSTTDYSSFMSPNKDLEKEKKFSFHRENYKKFVEASKPYMRYYGKVFIRDVKKSKLSTGKGEKIEVMYRSDEEIFTIFQILSYVRQSIMHNDIGNKSSILAIEKYPARFVGFLSDLLKTKTNDVNRMFIDNNSQTNFWVLFSIFGLQDHTSGADKICRNFYDFVIKADSKNLGFSLKKIRELMLDLPNANMLRDHQFDTVRSKFYTLLDFIIYQHYLEEKSRIDNMVEKLRMTLKEEEKEVLYAAEAKIVWNAIGAKVINKLVPMMNGDALKEIKRKNRDRKLPQSVIATVQVNSDANVFSGLIYFLTLFLDGKEINEMVSNLITKFENIDSLLHVDREIYKSDEKDLDLEIEKLALFFKGVVRPNAKTDTGAGEISKSFSIFQSAERIIEELKFIKNVTRMDNEIFPSEGVFLDAANVLGVRGDDFDFSNEFVGDDLHSDANKKIINKINGTKEDRNLRNFIINNVVKSRRFQYIARHMNTHYVKQLANNETLNRFVLNKMGDAKIINRYYESISGNTPNIEVRSQIDYLVKRLRSFSFEDLNDVKQKVRPGTNESIEKEKKKALVGLCLTIQYLVYKNLVNINARYTTAFYCLERDSKLKGFGVDVWRDFESYTALTNHFIKEGYLPVRKAEILRANLKHLDCEDGFKYYRNQVTHLNAIRVAYKYINEIKSVHSYFALYHYIMQRHLYDSLQAKAKDSSGFVIDALKKSFEHKIYSKDLLHVLHSPFGYNTARYKNLSIEALFDKNESRPEVNPLSTND

SEQ ID NO: 7 - protein, 906 aa, synthetic construct
  REGION 1..906: note = Synthetic: P1E0Cas13d
MEREVKKPPKKSLAKAAGLKSTFVISPQEKELAMTAFGRGNDALLQKRIVDGVVRDVAGEKQQFQVQRQDESRFRLQNSRLADRTVTADDPLHRAETPRRQPLGAGMDQLRRKAILEQKYFGRTFDDNIHIQLIYNILDIHKMLAVPANHIVHTLNLLGGYGETDFVGMLPAGLPYDKLRVVKKKNGDTVDIKADIAAYAKRPQLAYLGAAFYDVTPGKSKRDAARGRVKREQDVYAILSLMSLLRQFCAHDSVRIWGQNTTAALYHLQALPQDMKDLLDDGWRRALGGVNDHFLDTNKVNLLTLFEYYGAETKQARVALTQDFYRFVVLKEQKNMGFSLRRLREELLKLPDAAYLTGQEYDSVRQKLYMLLDFLLCRLYAQERADRCEELVSALRCALSDEEKDTVYQAEAAALWQALGDTLRRKLLPLLKGKKLQDKDKKKSDELGLSRDVLDGVLFRPAQQGSRANADYFCRLMHLSTWFMDGKEINTLLTTLISKLENIDSLRSVLESMGLAYSFVPAYAMFDHSRYIAGQLRVVNNIARMRKPAIGAKREMYRAAVVLLGVDSPEAAAAITDDLLQIDPETGKVRPRSDSARDTGLRNFIANNVVESRRFTYLLRYMTPEQARVLAQNEKLIAFVLSTVPDTQLERYCRTCGREDITGRPAQIRYLTAQIMGVRYESFTDVEQRGRGDNPKKERYKALIGLYLTVLYLAVKNMVNCNARYVIAFYCRDRDTALYQKEVCWYDLEEDKKSGKQRQVEDYTALTRYFVSQGYLNRHACGYLRSNMNGISNSLLTAYRNAVDHLNAIPPLGSLCRDIGRVDSYFALYHYAVQQYLNGRYYRKTPREQELFAAMAQHRTWCSDLVKALNTPFGYNLARYKNLSIDGLFDREGDHVVREDGEKPAE

SEQ ID NO: 8 - protein, 954 aa, synthetic construct
  REGION 1..954: note = Synthetic: EsCas13d
MGKKIHARDLREQRKTDRTEKFADQNKKREAERAVPKKDAAVSVKSVSSVSSKKDNVTKSMAKAAGVKSVFAVGNTVYMTSFGRGNDAVLEQKIVDTSHEPLNIDDPAYQLNVVTMNGYSVTGHRGETVSAVTDNPLRRFNGRKKDEPEQSVPTDMLCLKPTLEKKFFGKEFDDNIHIQLIYNILDIEKILAVYSTNAIYALNNMSADENIENSDFFMKRTTDETFDDFEKKKESTNSREKADFDAFEKFIGNYRLAYFADAFYVNKKNPKGKAKNVLREDKELYSVLTLIGKLRHWCVHSEEGRAEFWLYKLDELKDDFKNVLDVVYNRPVEEINNRFIENNKVNIQILGSVYKNTDIAELVRSYYEFLITKKYKNMGFSIKKLRESMLEGKGYADKEYDSVRNKLYQMTDFILYTGYINEDSDRADDLVNTLRSSLKEDDKTTVYCKEADYLWKKYRESIREVADALDGDNIKKLSKSNIEIQEDKLRKCFISYADSVSEFTKLIYLLTRFLSGKEINDLVTTLINKFDNIRSFLEIMDELGLDRTFTAEYSFFEGSTKYLAELVELNSFVKSCSFDINAKRTMYRDALDILGIESDKTEEDIEKMIDNILQIDANGDKKLKKNNGLRNFIASNVIDSNRFKYLVRYGNPKKIRETAKCKPAVRFVLNEIPDAQIERYYEACCPKNTALCSANKRREKLADMIAEIKFENFSDAGNYQKANVTSRTSEAEIKRKNQAIIRLYLTVMYIMLKNLVNVNARYVIAFHCVERDTKLYAESGLEVGNIEKNKTNLTMAVMGVKLENGIIKTEFDKSFAENAANRYLRNARWYKLILDNLKKSERAVVNEFRNTVCHLNAIRNININIKEIKEVENYFALYHYLIQKHLENRFADKKVERDTGDFISKLEEHKTYCKDFVKAYCTPFGYNLVRYKNLTIDGLFDKNYPGKDDSDEQK

SEQ ID NO: 9 - protein, 1094 aa, synthetic construct
  REGION 1..1094: note = Synthetic: PspCas13b
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMKGSLQ

SEQ ID NO: 10 - protein, 1179 aa, synthetic construct
  REGION 1..1179: note = Synthetic: PguCas13b
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKIDHEHNDEVDPHYHFNHLVRKGKKDRYGHNDNPSFKHHFVDGEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAERVQGRIKRVIEDVYAVYDAFARDEINTRDELDACLADKGIRRGHLPRQMIAILSQEHKDMEEKIRKKLQEMMADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENRPFLLLKEPKTDRQTLVAGWKGEFHLPRGIFTEAVRDCLIEMGHDEVASYKEVGFMAKAVPLYFERACEDRVQPFYDSPFNVGNSLKPKKGRFLSKEERAEEWERGKERFRDLEAWSYSAARRIEDAFAGIEYASPGNKKKIEQLLRDLSLWEAFESKLKVRADRINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQAGSLQ

SEQ ID NO: 11 - protein, 1095 aa, synthetic construct
  REGION 1..1095: note = Synthetic: RanCas13b
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQI

SEQ ID NO: 12 - protein, 411 aa, synthetic construct
  REGION 1..411: note = Synthetic: eIF4A3
MATTATMATSGSARKRLLKEEDMTKVEFETSEEVDVTPTFDTMGLREDLLRGIYAYGFEKPSAIQQRAIKQIIKGRDVIAQSQSGTGKTATFSISVLQCLDIQVRETQALILAPTRELAVQIQKGLLALGDYMNVQCHACIGGTNVGEDIRKLDYGQHVVAGTPGRVFDMIRRRSLRTRAIKMLVLDEADEMLNKGFKEQIYDVYRYLPPATQVVLISATLPHEILEMTNKFMTDPIRILVKRDELTLEGIKQFFVAVEREEWKFDTLCDLYDTLTITQAVIFCNTKRKVDWLTEKMREANFTVSSMHGDMPQKERESIMKEFRSGASRVLISTDVWARGLDVPQVSLIINYDLPNNRELYIHRIGRSGRYGRKGVAINFVKNDDIRILRDIEQYYSTQIDEMPMNVADLI

SEQ ID NO: 13 - protein, 174 aa, synthetic construct
  REGION 1..174: note = Synthetic: Y14/RBM8A (minimal sequence for NMD activation is underlined).
MADVLDLHEAGGEDFAMDEDGDESIHKLKEKAKKRKGRGFGSEEGSRARMREDYDSVEQDGDEPGPQRSVEGWILFVTGVHEEATEEDIHDKFAEYGEIKNIHLNLDRRTGYLKGYTLVEYETYKEAQAAMEGLNGQDLMGQPISVDWCFVRGPPKGKRRGGRRRSRSPDRRRR

SEQ ID NO: 14 - protein, 146 aa, synthetic construct
  REGION 1..146: note = Synthetic: MAGOH
MESDFYLRYYVGHKGKFGHEFLEFEFRPDGKLRYANNSNYKNDVMIRKEAYVHKSVMEELKRIIDDSEITKEDDALWPPPDRVGRQELEIVIGDEHISFTTSKIGSLIDVNQSKDPEGLRVFYYLVQDLKCLVFSLIGLHFKIKPI

SEQ ID NO: 15 - protein, 1118 aa, synthetic construct
  REGION 1..1118: note = Synthetic: UPF1
MSVEAYGPSSQTLTFLDTEEAELLGADTQGSEFEFTDFTLPSQTQTPPGGPGGPGGGGAGGPGGAGAGAAAGQLDAQVGPEGILQNGAVDDSVAKTSQLLAELNFEEDEEDTYYTKDLPIHACSYCGIHDPACVVYCNTSKKWFCNGRGNTSGSHIVNHLVRAKCKEVTLHKDGPLGETVLECYNCGCRNVFLLGFIPAKADSVVVLLCRQPCASQSSLKDINWDSSQWQPLIQDRCFLSWLVKIPSEQEQLRARQITAQQINKLEELWKENPSATLEDLEKPGVDEEPQHVLLRYEDAYQYQNIFGPLVKLEADYDKKLKESQTQDNITVRWDLGLNKKRIAYFTLPKTDSDMRLMQGDEICLRYKGDLAPLWKGIGHVIKVPDNYGDEIAIELRSSVGAPVEVTHNFQVDFVWKSTSFDRMQSALKTFAVDETSVSGYIYHKLLGHEVEDVIIKCQLPKRFTAQGLPDLNHSQVYAVKTVLQRPLSLIQGPPGTGKTVTSATIVYHLARQGNGPVLVCAPSNIAVDQLTEKIHQTGLKVVRLCAKSREAIDSPVSFLALHNQIRNMDSMPELQKLQQLKDETGELSSADEKRYRALKRTAERELLMNADVICCTCVGAGDPRLAKMQFRSILIDESTQATEPECMVPVVLGAKQLILVGDHCQLGPVVMCKKAAKAGLSQSLFERLVVLGIRPIRLQVQYRMHPALSAFPSNIFYEGSLQNGVTAADRVKKGFDFQWPQPDKPMFFYVTQGQEEIASSGTSYLNRTEAANVEKITTKLLKAGAKPDQIGIITPYEGQRSYLVQYMQFSGSLHTKLYQEVEIASVDAFQGREKDFIILSCVRANEHQGIGFLNDPRRLNVALTRARYGVIIVGNPKALSKQPLWNHLLNYYKEQKVLVEGPLNNLRESLMQFSKPRKLVNTINPGARFMTTAMYDAREAIIPGSVYDRSSQGRPSSMYFQTHDQIGMISAGPSHVAAMNIPIPFNLVMPPMPPPGYFGQANGPAAGRGTPKGKTGRGGRQKNRFGLPGPSQTNLPNSQASQDVASQPFSQGALTQGYISMSQPSQMSQPGLSQPELSQDSYLGDEFKSQIDVALSQDSTYQGERAYQHGGVTGLSQY

SEQ ID NO: 16 - protein, 1181 aa, synthetic construct
  REGION 1..1181: note = Synthetic: UPF2 (minimal sequence for NMD activation is underlined).
MDHPEDESHSEKQDDEEALARLEEIKKSIEAKLTLRQNNLNPERPDSAYLRTLDSSIKRNTAVIKKLKQINEEQREGLMDDLRGVNLSKFVSEAVTAICEAKLKSSDIQAAVQICSLLHQRYKEFSASLTQGLLKVFFPGKSAEDLEADKNSKAMKKRSTLKLLLELYYVGVIEDSNIFINIIKDLTSVEQLKDRDTTQTNLTLLTSFARQGRIFLGLPISGQDEDFFKGLDVTADQKKSFKKAFNTYYDALADLLQSEHKLLLQMEKENAKLVNAKGELSEDSASSYEKLRKSYDHLYRNISSLAEALDMQPPVMPEDGTTRLTAGDEASPSGTVKDTSVPEPIWDDEDTKTFYECLPDLRAFVPAVLLGEAEPKSNEQSAKAKEKLSESSSEVVENQQTTEDTTEVSADSASMDDRSNAEQPKEKEEVEKEKAKDTKKEKGKEKDSEKKMEHEKEKGKSLDVANFERLLQRLPGCVSRDLIDQLTVEYCYLNSKTNRKKLVKALFNVPRTSLELLAYYSRMVATLASCMKDIPSMLVQMLEDEFNSLVHKKDQMNIETKIRNIRFIGELCKFKIVPAGLVFSCLKACLDEFTHHNIDVACNLLETCGRFLYRSPETTLRMTNMLDILMRLKNVKNLDPRQSTLVENAYYLCKPPERSARISKVRPPLHQYVRKLLFSDLDKDSIANVLKQLRKLPWSECEQYILKCFMKVHKGKYGQIHLIASLTSGLSRHHDEFVVAVVDEVLEEIRVGLELNEYGAQQKRLAHMRFLGELYNYEHVDSSVIFETLYLTLLYGHDTSEQEVLDPPEDFFRVRMVIILLETCGHYFDRGSSKKRLDQFLIHFQRYILSKGHLPLDIEFDLQDLFANLRPNMTRYSTIDEVNAAILQLEEREHASSGDKVSIERHSDTKPSNKSSSDVISSNGKSTAKDIRENGEAHGEESDSDSGSGSVVRDGQNEELDDGNHERGSESGDGDDYDDGDGPGSDDDKFRVRQKVVTVDLEEQADFDQELKALLQESMEQRKLELRGRPALNMTIPMSVFEGSGKDHHHFGRVVGENGEEVLDEENGEQREVQVKVLVKRGNKQQTRQMLIPSDCALVQSTKQKEAAELEEKQDIKRLVLEYNERDEEEANGLGTQILNWTSGGSRGSTRTGEGSGKSGGSRHRFYYHQGGGGSYHARRK

SEQ ID NO: 17 - protein, 483 aa, synthetic construct
  REGION 1..483: note = Synthetic: UPF3B/3X
MKEEKEHRPKEKRVTLLTPAGATGSGGGTSGDSSKGEDKQDRNKEKKEALSKVVIRRLPPTLTKEQLQEHLQPMPEHDYFEFFSNDTSLYPHMYARAYINFKNQEDIILFRDRFDGYVFLDNKGQEYPAIVEFAPFQKAAKKKTKKRDTKVGTIDDDPEYRKFLESYATDNEKMTSTPETLLEEIEAKNRELIAKKTTPLLSFLKNKQRMREEKREERRRREIERKRQREEERRKWKEEEKRKRKDIEKLKKIDRIPERDKLKDEPKIKVHRFLLQAVNQKNLLKKPEKGDEKELDKREKAKKLDKENLSDERASGQSCTLPKRSDSELKDEKPKRPEDESGRDYREREREYERDQERILRERERLKRQEEERRRQKERYEKEKTFKRKEEEMKKEKDTLRDKGKKAESTESIGSSEKTEKKEEVVKRDRIRNKDRPAMQLYQPGARSRNRLCPPDDSTKSGDSAAERKQESGISHRKEGGEE

SEQ ID NO: 18 - protein, 33 aa, synthetic construct
  REGION 1..33: note = Synthetic: Nuclear Export Signal
NVKKRKRRSKKINKLTDGQIRINHVSSEKKRRE

SEQ ID NO: 19 - protein, 11 aa, synthetic construct
  REGION 1..11: note = Synthetic: Nuclear Export Signal
LQLPPLERLTL

SEQ ID NO: 20 - RNA, 30 nt, synthetic construct
  misc_feature 1..30: note = Synthetic: Exemplary hairpin sequence of guide RNA
  source 1..30: mol_type = other RNA
cactagtgcgaatttgcactagtctaaaac

SEQ ID NO: 21 - DNA, 23 bp, synthetic construct
  misc_feature 1..23: note = Synthetic: exemplary guide RNA sequence
  source 1..23: mol_type = other DNA
tggttcactctgaacggagctgg

SEQ ID NO: 22 - skipped

SEQ ID NO: 23 - protein, 7 aa, synthetic construct
  REGION 1..7: note = Synthetic: Exemplary linker sequence
GSGGGGS

SEQ ID NO: 24 - protein, 15 aa, synthetic construct
  REGION 1..15: note = Synthetic: Exemplary linker sequence
GGGGSGGGGSGGGGS

SEQ ID NO: 25 - protein, 18 aa, synthetic construct
  REGION 1..18: note = Synthetic: Exemplary linker sequence
GGSGGSGGSGGSGGSGGS

SEQ ID NO: 26 - protein, 32 aa, synthetic construct
  REGION 1..32: note = Synthetic: Exemplary linker sequence
SGGSSGGSSGSETPGTSESATPESSGGSSGGS

SEQ ID NO: 27 - protein, 305 aa, synthetic construct
  REGION 1..305: note = Synthetic: RNPS1
MDLSGVKKKSLLGVKENNKKSSTRAPSPTKRKDRSDEKSKDRSKDKGATKESSEKDRGRDKTRKRRSASSGSSSTRSRSSSTSSSGSSTSTGSSSGSSSSSASSRSGSSSTSRSSSSSSSSGSPSPSRRRHDNRRRSRSKSKPPKRDEKERKRRSPSPKPTKVHIGRLTRNVTKDHIMEIFSTYGKIKMIDMPVERMHPHLSKGYAYVEFENPDEAEKALKHMDGGQIDGQEITATAVLAPWPRPPPRRFSPPRRMLPPPPMWRRSPPRMRRRSRSPRRRSPVRRRSRSPGRRRHRSRSSSNSSR

SEQ ID NO: 28 - protein, 248 aa, synthetic construct
  REGION 1..248: note = Synthetic: SRSF1
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

(19) United States
(12) Patent Application Publication
(10) Pub. No.: US 2025/0197824 A1
(43) Pub. Date: Jun. 19, 2025

(54) RNA TARGETING COMPOSITIONS AND METHODS OF USE THEREOF

(71) Applicant: UNIVERSITY OF ROCHESTER, Rochester, NY (US)

(72) Inventors: Tatsuaki KUROSAKI, Rochester, NY (US); Lynne MAQUAT, Rochester, NY (US)

(21) Appl. No.: 18/847,015

(22) PCT Filed: May 4, 2023
(86) PCT No.: PCT/US2023/066588

§ 371 Date: Sep. 13, 2024

Related U.S. Application Data
(60) Provisional application No. 63/364,237, filed on May 5, 2022.

(51) Int. Cl. C12N 9/22; C07K 14/47; C12N 15/11

(52) U.S. Cl. CPC C12N 9/22; C07K 14/47; C12N 15/11; C07K 2319/095; C12N 2310/20

(57) ABSTRACT

An artificial exon-junction complex comprising: a fusion protein comprising a RNA binding domain and an exon-junction complex (EJC) mimicking domain; a guide RNA; and a target mRNA wherein the RNA binding domain comprises a RNA binding region of a CAS nuclease, wherein the (EJC) mimicking domain comprises one or more regions that are derived from a component of EJC, wherein the guide RNA comprises a sequence complementary to a target sequence in the target mRNA, wherein the target mRNA comprises a premature termination codon (PTC) that is insensitive to nonsense-mediated mRNA decay (NMD), and wherein 5′ end of the target sequence is located at least 10 nucleotides downstream from the PTC in the target mRNA. A method for inducing mRNA decay of a target mRNA comprising a premature termination codon (PTC) that is insensitive to the nonsense-mediated mRNA decay (NMD) process.

This application claims priority to U.S. Provisional Patent Application No. 63/364,237, filed May 5, 2022.

This invention was made with government support under GM147719 and GM059614 awarded by the National Institutes of Health. The government has certain rights in the invention.

### FIELD

This application relates generally to the field of medical treatment and, in particular, to compositions that specifically target mRNAs.

### BACKGROUND

All cellular processes are not without errors. Accordingly, cells have evolved complex quality-control mechanisms aimed at dealing with its own mistakes. One particularly prevalent and deleterious error is the introduction of a premature termination codon (PTC) within a protein encoding exon. The resulting PTC-containing transcripts are best recognized and eliminated before the truncated proteins that they encode accumulate in the cell. Failure to do so can have pathological consequences in humans, as evidenced by the fact there are many dominantly inherited diseases that are due to a PTC that fails to trigger NMD. Notably, about 33% of inherited and acquired diseases are a result of PTC acquisition.

Nonsense-mediated mRNA decay (NMD) is a cellular mechanism that selectively degrades mRNAs harboring a PTC. The NMD process, however, is only effective in eliminating mRNAs with PTCs situated more than ˜55-nts upstream of the last exon-exon junction (FIG. 1). mRNAs harboring PTCs that are located in the last exon or less than 55-nts upstream of the last exon-exon junction are resistant to the NMD process and are the main cause of dominantly inherited diseases that are due to a PTC.

Nonsense-mediate mRNA decay (NMD) is an essential cellular process that ensures the proper regulation of gene expression, allows cells to respond to environmental changes, maintains mRNA quality, and prevents the development of diseases. After pre-mRNA splicing, newly synthesized mRNAs are rapidly exported to the cytoplasm and translated. When translation terminates at a premature termination codon (PTC) located more than 55 nucleotides upstream of an exon-exon junction, the ribosome will not dislocate the downstream exon-junction complex (3′UTR EJC) (FIG. 2). In such cases, the 3′UTR EJC interact with the central NMD factor UPF1, and the complex of serine/threonine kinases SMG1-SMG8-SMG9 phosphorylate UPF1 (FIG. 2). UPF1 phosphorylation triggers mRNA decay, achieved by recruiting nucleases directly, as in the case of endonucleolytic decay by SMG6, or indirectly, as in the case of exonucleolytic decay through the SMG5-SMG7 heterodimer followed by the CCR4-NOT deadenylation complex to complete the subsequent round of mRNA decay (FIG. 2). Since a 3′UTR EJC is a canonical activator of NMD, most of the 3′UTR EJC-containing mRNAs are rapidly degraded within a minute of export to the cytoplasm.

Accordingly, there exists a need for compositions and methods that may effectively treat conditions caused by NMD-insensitive PTCs.

### SUMMARY

One aspect of the present application relates to a fusion protein that comprises a RNA binding domain and an exon-junction complex (EJC) mimicking domain, wherein the RNA binding domain comprises a Cas nuclease or a RNA binding region thereof and wherein the EJC mimicking domain comprises one or more regions derived from one or more components of EJC.

In some embodiments, the fusion protein further comprises at least one nuclear export signal. In some embodiments, the nuclear export signal comprises SEQ ID NO:18 or 19.

In some embodiments, the RNA binding domain comprises a catalytically inactive Cas nuclease or a RNA binding region thereof. In some embodiments, the RNA binding domain comprises a catalytically inactive Cas13d nuclease or a RNA binding region thereof

In some embodiments, the component of the EJC is selected from the group consisting of elF4A3, Y14/RBM8A, MAGOH, RNPS1, SRSF1, UPF1, UPF2 and UPF3B/3X

In some embodiments, the fusion protein further comprises a linker between the RNA binding domain and the EJC mimicking domain.

Another aspect of the present application relates to an expression vector that comprises a first nucleotide sequence encoding a RNA binding domain of a CAS nuclease, a second nucleotide sequence encoding an exon-junction complex (EJC) mimicking domain derived from a component of EJC, and a regulatory sequence operably linked to at least one of the first and the second nucleotide sequence.

In some embodiments, the expression vector further comprises a nucleotide sequence encoding a guide RNA. In some embodiments, the guide RNA comprises a sequence complementary to a target sequence in a NMD-insensitive PTC-containing mRNA, wherein the 5′ end of the target sequence is located at least 10 nucleotides downstream from the NMD-insensitive PTC in the mRNA. In some embodiments, the expression vector is a non-viral vector. In some embodiments, the expression vector is a viral vector. In some embodiments, the expression vector is an AAV vector.

Another aspect of the present application relates to a pharmaceutical composition comprising the expression vector of the present application and a pharmaceutically acceptable carrier.

Another aspect of the present application relates to an artificial exon-junction complex that comprises a fusion protein comprising a RNA binding domain and an exon-junction complex (EJC) mimicking domain, a guide RNA and a target mRNA, wherein the RNA binding domain comprises a RNA binding region of a CAS nuclease, wherein the (EJC) mimicking domain comprises one or more regions that are derived from a component of EJC, wherein the guide RNA comprises a sequence complementary to a target sequence in the target mRNA, wherein the target mRNA comprises a premature termination codon (PTC) that is insensitive to nonsense-mediated mRNA decay (NMD), and wherein the 5′ end of the target sequence is located at least 10 nucleotides downstream from the PTC in the target mRNA. In some embodiments, the fusion protein further comprises a nuclear export signal.

Another aspect of the present application relates to a method for treating a disease or a condition in a subject caused by translation of a target mRNA comprising a premature termination codon (PTC) that is insensitive to the nonsense-mediated mRNA decay (NMD) process. The method comprises the steps of introducing into cells of the subject expressing the target mRNA (1) a fusion protein comprising a RNA binding domain and an exon-junction complex (EJC) mimicking domain and (2) a guide RNA that binds specifically to a target region in the target mRNA, wherein the fusion protein and the guide RNA form a complex that binds to the target mRNA and results in the degradation of the target mRNA by NMD. In some embodiments, the fusion protein is introduced into the cells in the form of one or more expression vectors that express the fusion protein. In some embodiments, the expression vectors further express the guide RNA.

Another aspect of the present application relates to a method for inducing mRNA decay of a target mRNA comprising a premature termination codon (PTC) that is insensitive to the nonsense-mediated mRNA decay (NMD) process, the method comprises the steps of introducing into cells expressing the target mRNA (a) an expression vector comprising: a first nucleotide sequence encoding a RNA binding domain; a second nucleotide sequence encoding an exon-junction complex (EJC) mimicking domain derived from one or more components of the EJC; and a regulatory sequence operably linked to at least one of the first and the second nucleotide sequence; and (b) a guide RNA that binds specifically to a target region in the target mRNA, wherein the expression vector expresses a fusion protein comprising a RNA binding domain and the EJC mimicking domain in the cells.

### BRIEF DESCRIPTION OF THE DRAWINGS

FIG. 1 shows the location of the NMD-sensitive PTC region (blue) and the NMD-insensitive PTC region (red) in a mRNA.

FIG. 2 is a diagram showing the molecular mechanism of NMD, displaying one of several EJC compositions that are possible and not showing SMG6-mediated endonucleolytic cleavage of the mRNA.

FIG. 3 shows an embodiment of RNA-Programmed NMD Activation (RP-NMDA). The upper panel shows dCas13d-mediated EJC tethering on a normal mRNA. This mRNA will function normally since translating ribosomes will remove the tethered protein(s). The lower panel shows dCas13d-mediated EJC tethering to, and the subsequent degradation of, the NMD-insensitive PTC-containing counterpart of the normal mRNA.

FIG. 4 is a diagram showing human beta-globin (HBB) reporter mRNAs, EGFP-HBB-121Ter and mRFP-HBB-Norm, showing the NMD-insensitive PTC at HBB codon 121 (121Ter) and the normal termination codon (Norm Ter). Exon-exon junctions are shown in black vertical lines. The guide RNA target region on EGFP-HBB-121Ter mRNA is shown in a horizontal light blue line.

FIG. 5 is a diagram showing APC reporter transcripts. FLAG-tagged normal APC (FLAG-APC Norm), APC harboring a PTC at codon 1309 (FLAG-APC 1309Ter), and APC 1309 Ter harboring 3′-intron (FLAG-APC 1309Ter+intron) are shown. Nucleotides encoding the mutation cluster region (MCR) are indicated as a blue line under the FLAG-APC Norm transcript. The NMD-insensitive PTC region is highlighted in cyan.

FIG. 6 shows NMD-sensitive or insensitive nonsense mutations on HBB reporter constructs. (Panel A) Diagram of human beta-globin (HBB) reporter construct containing a normal termination codon (NormTer), NMD-sensitive (39Ter), or NMD-insensitive PTC (121Ter and 127Ter). EGFP mRNA serves as a transfection control. (Panel B) Western blot analysis showing protein expressions of specified HBB constructs. (Panel C) RT-qPCR analysis showing relative mRNA abundance of specified HBB construct. The data represent means with s.d. (n=3). *** P<0.001 (student t-test).

### DETAILED DESCRIPTION

Herein incorporated by reference is the sequence listing filed with the USPTO as 1134-107 PCT.xml which was created on May 4, 2023, and the size is 49,906 bytes.

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

Remarkably, if the 3′UTR EJC mimic is reconstituted downstream of a normal termination codon, NMD can be induced. By using a bacteriophage MS2 coat protein and a lambda bacteriophage anti-terminator protein N-BoxB system, tethering the EJC constituent Y14 or EJC associating factors such as UPF3B/3X, but not cytoplasmic poly A binding protein (PABPC1) sufficiently (>˜20-24 nt) downstream of termination codon transforms a non-NMD substrate to be an NMD target. Therefore, the molecular tethering method, which eventually utilizes the CRISPR-Cas system instead of the bacteriophage MS2 coat protein and a lambda bacteriophage anti-terminator protein N-BoxB system, is feasible for activating NMD for any NMD-insensitive transcripts.

### I. Definitions

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” are also disclosed, as appropriately understood by the skilled artisan. For example, if the value “10” is disclosed, the “less than or equal to 10” and “greater or equal to 10” is also disclosed. When two or more value are disclosed, all possible ranges between any two values are disclosed.

As used herein, the term “fusion protein” refers to proteins that are created through the joining of two or more protein domains that originally formed components of separate proteins.

As used herein, the term “exon-junction complex (EJC)” is a protein complex which forms on a pre-messenger RNA strand upstream of the junction of two exons that have been joined together during pre-mRNA splicing. The EJC is normally deposited about 20 nt-24 nt upstream of the junction and has major influences on translation, surveillance and localization of the spliced mRNA. It is first deposited onto pre-mRNA during splicing and is then transported on the spliced mRNA into the cytoplasm. There it plays a major role in post-transcriptional regulation of mRNA. It is believed that exon-junction complexes provide a position-specific memory of the splicing event as well as a binding platform for factors involved in mRNA export and nonsense-mediated mRNA decay. The EJC consists of a stable heterotrimer core, which serves as a binding platform for other factors necessary for the mRNA pathway. The core of the EJC contains the protein eukaryotic initiation factor 4A3 (eIF4A3; a DEAD-box RNA helicase) bound to an adenosine triphosphate (ATP) analog, as well as the additional proteins MAGOH and Y14/RBM8A. Exon-junction complexes play a major role in mRNA surveillance. More specifically, they are function in the nonsense mediated decay pathway (NMD), wherein mRNA transcripts with premature stop codons are degraded. In the translation of most normal mRNAs, i.e. mRNAs that are not NMD targets, the ribosome binds to the transcript and begins amino acid chain elongation. It continues on until it reaches the location of an exon junction complex, which it then displaces. This process is repeated until all exon-junction complexes are displaced. Next, translation is complete when the ribosome reaches a termination codon. The EJC and its position serve as a type of regulator, determining whether the transcript should be targeted for NMD or not.

As used herein, the term “expression” is defined as the transcription and/or translation of a particular nucleotide sequence driven by a regulatory sequence such as a promoter and/or an enhancer.

As used herein, the term “expression vector” refers to a composition of matter which comprises a nucleotide sequence encoding a protein and/or an RNA and which can be used to deliver the nucleic acid sequence to the interior of a cell and express the encoded protein and/or RNA inside the cell. An expression vector typically comprises a regulatory sequence for expression of the protein or RNA encoded by the nucleotide sequence.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “nucleotide sequence” includes all nucleotide sequences that are degenerate versions of each other and that encode the same amino acid sequence. Nucleotide sequences that encode proteins and RNA may include introns.

As used herein, the term “regulatory sequence” means a nucleic acid sequence which is required for expression of a coding sequence (either for protein or RNA) operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner. The term “promoter” as used herein is defined as a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

When in reference to nucleobases, the terms “nucleobase complementarity” and “complementarity” refer to a nucleobase that is capable of base pairing with another nucleobase. For example, in DNA, adenine (A) is complementary to thymine (T). For example, in RNA, adenine (A) is complementary to uracil (U). Complementarity can be partial or total. Partial complementarity occurs when one or more nucleic acid bases is not matched according to the base pairing rules. Total or complete complementarity between nucleic acids occurs when each and every nucleic acid base is matched with another base under the base pairing rules. In certain embodiments, a complementary nucleobase refers to a nucleobase of an antisense oligonucleotide that is capable of base pairing with a nucleobase of its target nucleic acid. For example, if a nucleobase at a certain position of an antisense oligonucleotide is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, to “treat” a disease or condition, means to reduce or eliminate a sign or symptom of the disease or condition, to stabilize the disease or condition, and/or to reduce or slow further progression of the disease or condition. In some embodiments, “treat”, “treatment” or “treating” is intended to include prophylaxis, amelioration, prevention or cure from the disease or condition.

As used herein, the term “effective amount” is an amount necessary or sufficient to achieve the desired biological effect or the selected result, and such amount can be determined by one of ordinary skill in the art as a matter of routine experimentation.

As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents.

### II. Compositions of the Present Application

The present application describes a novel approach to induce nonsense-mediated mRNA decay (NMD) of a PTC-containing mRNA that is resistant to NMD (also referred to as “NMD-insensitive PTC-containing mRNA” or “target RNA”) by forming an artificial exon-junction complex (EJC) on the target RNA. The formation of the artificial EJC would then lead to the degradation of the target RNA by NMD. This approach may be used for treating diseases and conditions caused by a NMD insensitive PTC-containing mRNA.

### A. Fusion Proteins

One aspect of the present application relates to a fusion protein that comprises (1) an RNA binding domain and (2) an exon-junction complex (EJC) mimicking domain. The fusion protein of the present application is capable of binding to a NMD-insensitive PTC-containing mRNA in the presence of a guide RNA by forming an artificial exon-junction complex (EJC) on the NMD-insensitive PTC-containing mRNA. In some embodiments, the fusion protein further comprises at least one nuclear exporting signal. In some embodiments, the fusion protein further comprises a linker.

### RNA Binding Domain

In some embodiments, the RNA-binding domain comprises the complete Cas nuclease or the RNA-binding region/sequence of a Cas nuclease. As used herein, the term “Cas nuclease” refers to a CRISPR-associated (Cas) nuclease which in its active state is capable of cleaving the phosphodiester bonds between nucleotides of nucleic acids. As used herein, the term “Cas nuclease” also includes variants of a Cas nuclease, such as a catalytically inactive Cas nuclease, or an RNA binding region/sequence of a Cas nuclease.

Examples of Cas nucleases include, but are not limited to: Cas9, Cas12 and Cas13. In some embodiments, the RNA-binding domain comprises the complete protein region or the RNA binding region/sequence of a nuclease of the Cas13 family.

In some embodiments, the RNA-binding domain comprises the complete protein region or the RNA-binding region/sequence of a Cas nuclease that does not require the protospacer flanking sequence (PFS) for Cas targeting.

In some embodiments, the RNA-binding domain comprises the complete protein region or the RNA-binding region/sequence of a Cas13d nuclease or a variant thereof. Examples of Cas13d include, but are not limited to: Ruminoccocus flavefaciens XPD3002 (RfxCas13d) (SEQ ID. NO: 1), Uncultured Ruminoccocus sp. (Ur) Cas13d (SEQ ID. NO: 3), Ruminoccocus flavefaciens FD1 (Rff) Cas13d (SEQ ID. NO: 4), Ruminoccocus albus (Ra) Cas13d (SEQ ID. NO: 5), Anaerobic digester metagenome 15706 (Adm) Cas13d (SEQ ID. NO: 6), Gut metagenome assembly PIE0-k21 (PIE0) Cas13d (SEQ ID. NO: 7), Eubacterium siraeum DSM15702 (Es) Cas13d (SEQ ID. NO: 8).

In some embodiments, the RNA-binding domain comprises the complete protein region or the RNA-binding region/sequence of a Cas13b nuclease or a variant thereof. Examples of Cas13b nucleases include, but are not limited to, Prevotella sp. P5-125 (Psp) Cas13b (SEQ ID. NO: 9), Porphyromonas gulae (Pgu) Cas13b (SEQ ID. NO: 10), Riemerella anatipestifer (Ran) Cas13b (SEQ ID. NO: 11).

In some embodiments, the RNA-binding domain comprises a catalytically inactive Cas nuclease, which does not cleave RNA bonds, but retains specific single-stranded RNA binding property. In some embodiments, the catalytically inactive Cas nuclease is a catalytically inactive Cas13d (dCas13d). In some embodiments, the catalytically inactive Cas nuclease is dRfxCas13d (SEQ ID. NO: 2), which harbors four positively charges amino acid substitutions (R295A, H300A, R849A and H854A).

One of ordinary skill will understand that the present application includes all suitable Cas nuclease RNA-binding domains that are capable of binding to a guide RNA to form a fusion protein/guide RNA complex that, when binds to a target mRNA, is capable of initiating NMD of the target mRNA.

### EJC Mimicking Domain.

The EJC-mimicking domain can be any protein domain that, when the fusion protein of the present application binds to a target RNA, is capable of forming a EJC or having the functional properties of a EJC that initiates NMD of the target RNA. In some embodiments, the EJC-mimicking domain comprises the complete or a partial region/sequence of one or more EJC proteins.

As used herein, the term “EJC proteins” include, but are not limited to, at least one of eIF4A3, Y14/RBM8A, MAGOH, RNPS1, SRSF1, and EJC-associating NMD factors such as UPF1, UPF2 and UPF3X/3B.

In some embodiments, the EJC-mimicking domain comprises the complete or a partial region/sequence of one or more of eIF4A3, Y14/RBM8A and MAGOH. In some embodiments, EJC-mimicking domain comprises the complete or a partial region of one or more EJC proteins selected from the group consisting of elF4A3 (SEQ ID. NO: 12), Y14/RBM8A (SEQ ID. NO: 13), MAGOH (SEQ ID. NO: 14), RNPS1 (SEQ ID. NO: 27), SRSF1 (SEQ ID. NO: 28), UPF1 (SEQ ID. NO: 15), UPF2 (SEQ ID. NO: 16) and UPF3X/3B (SEQ ID. NO: 17).

In some embodiments, the EJC-mimicking domain comprises the complete or a partial region of one or more of eIF4A3 (SEQ ID. NO: 12), Y14/RBM8A (SEQ ID. NO: 13) and MAGOH (SEQ ID. NO: 14).

### Nuclear Exporting Signal

As used herein, the term “nuclear exporting signal” is a short target peptide containing at least four hydrophobic residues in a protein that targets that protein for export out of the cell nucleus to the cytoplasm through the nuclear pore complex using nuclear transport.

In some embodiments, the fusion protein includes at least one nuclear exporting signal. In some embodiments, the nuclear-exporting signal is attached to the N-terminal or C-terminal end of the fusion protein of the present application. The nuclear-exporting signal localizes the protein to the cytoplasm for targeting cytoplasmic RNA. In some embodiments, the nuclear-exporting signal comprises an amino acid sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO:15 or 16. In some embodiments, the nuclear-exporting signal comprises an amino acid sequence of SEQ ID NO: 18 or 19.

### Linker

In some embodiments, the RNA-binding domain is joined to the EJC mimicking domain directly. In other embodiments, the RNA-binding domain is joined to the EJC-mimicking domain by a peptide linker of 2-40 amino acid residues. The linker forms a physical connection between two protein domains without interfering with the functioning of either protein domain. In some embodiments, the linker comprises hydrophilic residues. In some embodiments, the linker is the remainder resulting from the restriction cloning used to generate the fusion. Examples of the linker sequences include, but are not limited to, GS (SEQ ID NO: 22), GSGGGGS (SEQ ID NO: 23), GGGGSGGGGGGGGS (SEQ ID NO: 24), GGSGGSGGSGGSGGSGGS (SEQ ID NO: 25), SGGSSGGSSGSETPGTSESATPESSGGSSGGS (SEQ ID NO: 26).

### B. Expression Vectors

Another aspect of the present application relates to an expression vector that is capable of expressing the fusion protein of the present application in a cell. In some embodiments, the expression vector comprises a first nucleotide sequence encoding an RNA-binding domain; a second nucleotide sequence encoding an EJC-mimicking domain; and a regulatory sequence operably linked to at least one of the first and the second nucleotide sequence.

In some embodiments, the first nucleotide sequence encodes a complete Cas13b nuclease or a RNA-binding region thereof or a variant thereof.

In some embodiments, the first nucleotide sequence encodes a complete protein region or the RNA-binding region/sequence of a Cas13d nuclease or a variant thereof.

In some embodiments, the first nucleotide sequence encodes a complete protein region or the RNA-binding domain/sequence of a catalytically inactive Cas13d (dCas13d).

In some embodiments, the catalytically inactive Cas nuclease is dRfxCas13d (SEQ ID. NO: 2), which harbors four positively charges amino acid substitutions (R295A, H300A, R849A and H854A).

In some embodiments, the second nucleotide sequence encodes an EJC-mimicking domain comprising the complete or a partial region/sequence of one or more EJC proteins.

In some embodiments, the second nucleotide sequence encodes the EJC-mimicking domain comprising the complete or a partial region/sequence of one or more of eIF4A3, Y14/RBM8A and MAGOH. In some embodiments, EJC-mimicking domain comprises the complete protein region or a partial region/sequence of one or more EJC proteins selected from the group consisting of elF4A3 (SEQ ID. NO: 12), Y14/RBM8A (SEQ ID. NO: 13), MAGOH (SEQ ID. NO: 14), RNPS1 (SEQ ID. NO: 27), SRSF1 (SEQ ID. NO: 28), UPF1 (SEQ ID. NO: 15), UPF2 (SEQ ID. NO: 16) and UPF3X/3B (SEQ ID. NO: 17).

In some embodiments, the second nucleotide sequence encodes the EJC-mimicking domain comprising the complete protein region or a partial region of one or more EJC proteins selected from the group consisting of elF4A3 (SEQ ID. NO: 12), Y14/RBM8A (SEQ ID. NO: 13), MAGOH (SEQ ID. NO: 14), RNPS1 (SEQ ID. NO: 27), SRSF1 (SEQ ID. NO: 28), UPF1 (SEQ ID. NO: 15), UPF2 (SEQ ID. NO: 16) and UPF3X/3B (SEQ ID. NO: 17).

In some embodiments, the second nucleotide sequence encodes the EJC-mimicking domain comprising the complete protein region or a partial region of one or more of eIF4A3 (SEQ ID. NO: 12), Y14/RBM8A (SEQ ID. NO: 13) and MAGOH (SEQ ID. NO: 14).

In some embodiments, the expression vector further comprises a nucleotide sequence encoding a nuclear export signal. In some embodiments, the nuclear exporting signal comprises an amino acid sequence of SEQ ID NO:18 or 19.

In some embodiments, the expression vector further comprises a nucleotide sequence encoding a guide RNA. As used herein, the term “guide RNA” refers to an RNA that functions as a guide for the RNA-binding domain of the present application, so as to allow the fusion protein of the present application to bind to a NMD-insensitive PTC-containing mRNA to form an artificial exon-junction complex (EJC) on the NMD-insensitive PTC-containing mRNA. The formation of the artificial EJC results in the initiation of NMD and degradation of the NMD-insensitive PTC-containing mRNA.

In some embodiments, the guide RNA comprises a nucleotide sequence that is complementary to a target sequence in the NMD-insensitive PTC-containing mRNA. In some embodiments, the 5′ end of the target sequence is (1) at least 10, 15, 20, 25 or 30 nucleotides downstream of the NMD-insensitive PTC and (2) within the normal protein-coding sequence of the normal mRNA translation reading frame. As used herein, the term “normal mRNA” refers to a mRNA that does not contain the NMD-insensitive PTC, but otherwise is identical to the NMD-insensitive PTC-containing mRNA. The term “normal protein” refers to the protein product translated from the “normal mRNA.” In some embodiments, the 5′ end of the target sequence is at least 20-24 nucleotides downstream of the NMD-insensitive PTC.

In some embodiments, the target sequence comprises at least 15, 18 or 21 nucleotides to maintain binding specificity of the guide RNA. In some embodiments, the guide RNA comprises a hairpin structure. In some embodiments, the hairpin structure is 30-36 nt in length and contains an 8-10-nucleotide stem with A/U-rich loop. In some embodiments, the hairpin structure comprises the sequence of 5′-CACUAGUGCGAAUUUGCACUAGUCUAAAAC-3′ (SEQ ID NO:20).

The expression vectors can be any vector suitable for expression of the fusion protein and/or the guide RNA of the present application in eukaryotes. Typical expression vectors contain transcription and translation terminators, initiation sequences, and promoters useful for regulation of the expression of the desired nucleic acid sequence. In some embodiments, the one or more expression vectors are engineered to direct expression of the polynucleotide antagonist ubiquitously (constitutively). Exemplary promotors for ubiquitous expression, include, but are not limited to, the cytomegalovirus (CMV) immediate early promoter, an RSV LTR, a MoMLV LTR, a phosphoglycerate kinase-1 (PGK) promoter, a simian virus 40 (SV40) promoter, a CK6 promoter, a transthyretin promoter (TTR), a TK promoter, a tetracycline responsive promoter (TRE), a U6 promoter, an E2F promoter, a telomerase (hTERT) promoter, an H1 promoter, a cytomegalovirus enhancer/chicken beta-actin/rabbit β-globin promoter (CAG) promoter, an elongation factor 1-alpha promoter (EF1-α) promoter, a human β-glucuronidase promoter, a chicken β-actin (CBA) promoter, a retroviral Rous sarcoma virus (RSV) LTR promoter, a dihydrofolate reductase promoter, a β-actin promoter and the like.

In certain embodiments, the one or more expression vectors are engineered to provide tissue-specific expression of the polynucleotide to targeted cells, including a variety of B cell promoters known to be active in plasma cells, plasmablasts, lymphoplasmacytoid cells, memory B cells, B-2 cells, Follicular (FO) B cells, Marginal zone (MZ) B cells, B-1 cells, and regulatory B (Breg) cells

In some embodiments, the expression vector is a non-viral vector, such as a plasmid, a phagemid, or a cosmid. In some embodiments, the expression vector is a plasmid vector. Examples of plasmid vectors include, but are not limited to, pXR002: EF1a-dCasRx-2A-EGFP, pXR003: CasRx gRNA, pXR004: CasRx pre-gRNA, and derivative vectors.

In some embodiments, the expression vector is in the form of a recombinant virus (viral vector). The term “recombinant virus” or “viral vector” is used herein with reference to a virus that has been genetically altered, e.g., by the addition or insertion of a heterologous nucleic acid construct into a virus particle.

Viral vectors for expression of the polynucleotide antagonist may be derived from, e.g., adenoviruses, adeno-associated viruses (AAV), retroviruses (including lentiviruses, such as HIV-1 and HIV-2), vaccinia viruses and other poxviruses, herpesviruses (e.g., herpes simplex virus Types 1 and 2), polioviruses, Sindbis and other RNA viruses, alphaviruses, astroviruses, coronaviruses, orthomyxoviruses, papovaviruses, paramyxoviruses, parvoviruses, picornaviruses, togaviruses and others. The viral vectors may or may not contain sufficient viral genetic information and/or structural components for production of infectious virus when introduced into host cells, i.e., viral vectors may be replication-competent or replication-defective. In some embodiments, e.g., where structural components for production of infectious virus are lacking, the necessary functional components may be supplied in trans by a host cell or by another vector introduced into the cell when production of recombinant virus is desired. In preferred embodiments, replication-defective recombinant viruses are administered for treatment. A nucleic acid for delivery may be incorporated into a naturally occurring or modified viral genome (or a portion thereof) or may be present within a viral capsid as a separate nucleic acid molecule.

In some embodiments, the viral vectors may be engineered to target specific cells, such as plasma cells or multiple myeloma cells by using the targeting characteristics inherent to the virus vector or engineered into the virus vector. Specific cells may be “targeted” for delivery and expression of polynucleotides. Thus, “targeting,” in this case, relates to the use of endogenous or heterologous binding agents in the form of capsids, envelope proteins, antibodies for delivery to specific cells, the use of tissue-specific regulatory elements for restricting expression to specific subset(s) of cells, or both.

In some embodiments, the viral vectors are AAV vectors. AAV vectors provide a preferred delivery system for the nucleic acid therapeutics of the present application since they can allow for long lasting and continuous expression of the fusion protein and/or guide RNA of the present application. AAV vectors can include or can be modified to control expression of the fusion protein and/or the guide RNA under a number of different regulatory elements, including various promoter and/or enhancer elements for constitutive or cell-type specific expression.

Any suitable AAV serotype or AAV pseudotypes may be used to express the fusion protein and/or the guide RNA of the present application in cells in vivo. The types of vectors for in vivo delivery are preferably chosen based on lack of pre-existing immunity in the host to a selected AAV subtype and stable expression in vivo. Typically, AAV vectors are derived from single-stranded (ss) DNA parvoviruses that are nonpathogenic for mammals. Among the serotypes of AAVs isolated from human or non-human primates, human serotype 2 is the first and best characterized AAV that was developed as a gene transfer vector. Other useful AAV serotypes include AAV1, AAV3, AAV4, AAV5, AAV6, AAV7, AAV8, AAV9, AAV9.47, AAV9 (hu14), AAV10, AAV11, AAV12, AAVrh8, AAVrh10, AAV-DJ8 and AAV-DJ.

In some embodiments, the AAV vector may be a pseudotyped AAV vector containing sequences and/or components originating from at least two different AAV serotypes. Thus, a pseudotyped (or chimeric) AAV vector may include, for example, an AAV2-derived genome in an AAV1-derived or AAV6-derived capsid; or an AAV2-derived genome in an AAV4-derived capsid; or an AAV2-derived genome in an AAV9-derived capsid. Alternatively, a pseudotyped AAV vector may include a portion of the capsid from one AAV serotype fused to a second portion of a different AAV serotype capsid, resulting in a vector encoding a pseudotyped AAV2/AAV5 capsid. In other embodiments, a pseudotyped AAV vector may include a capsid from one serotype and inverted terminal repeats (ITRs) from another AAV serotype. Exemplary AAV vectors include recombinant pseudotyped AAV2/1, AAV2/2, AAV2/5, AAV2/7, AAV2/8, and AAV2/9 serotype vectors.

In some embodiments, the expression vector of the present application is an AAV vector produced from pAAV-hU6-DR30-Bsal_EFS-dCasRx-bghpA or a derivative vector.

### C. Pharmaceutical Compositions

Another aspect of the present application relates to a pharmaceutical composition that comprises an expression vector of the present application and a pharmaceutically acceptable carrier.

Exemplary carriers for delivery include buffers, nanoparticles, lipids, liposomes, micelles, polymers, polymeric micelles, emulsions, polyelectrolyte complexes, hydrogels, microcapsules, exosomes, combinations thereof, and pegylated derivatives thereof.

In some embodiments, the pharmaceutical composition comprises a viral expression vector and an aqueous buffer. Suitable buffers include but are not limited to, physiological saline buffer, Hanks's solution, Ringer's solution, sodium succinate buffer, sodium citrate buffer, sodium phosphate buffer or potassium phosphate buffer.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution.

In some embodiments, the pharmaceutical composition comprises a non-viral expression vector and a nanoparticle carrier. Exemplary nanoparticles include paramagnetic nanoparticles, superparamagnetic nanoparticles, metal nanoparticles, polymeric nanoparticles, nanoworms, nanoemulsions, nanogels, fullerene-like materials, inorganic nanotubes, dendrimers (such as with covalently attached metal chelates), nanocapsules, nanospheres, nanofibers, nanohoms, nano-onions, nanorods, nanoropes and quantum dots. A nanoparticle can produce a detectable signal, for example, through absorption and/or emission of photons (including radio frequency and visible photons) and plasmon resonance. Nanoparticles can be biodegradable or non-biodegradable. In some embodiments, the nanoparticle carrier is linked to a tissue-specific targeting peptides or antibodies to facilitate carrier-mediated delivery of the expression vectors.

In some embodiments, the pharmaceutical composition is in a lyophilized dosage form and comprise a cryoprotectant. Examples of cryoprotectants include, but are not limited to, sucrose (optimally 0.5-1.0%), trehalose and lactose. In some embodiments, the pharmaceutical composition further comprises a bulking agent. Examples of bulking agents include, but are not limited to, mannitol, glycine and arginine.

In some embodiments, the pharmaceutical composition of the present application is formulated for parenteral administration by injection, such as intravenous, intramyocardial, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The pharmaceutical forms suitable for injectable use include sterile aqueous solutions or dispersions and sterile powders for the extemporaneous preparation of sterile injectable solutions or dispersions. The form should be sterile and fluid to the extent that easy syringability exists. It should be stable under the conditions of manufacture and storage and should be preserved against the contaminating action of microorganisms, such as bacteria and fungi. The pharmaceutical carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants.

Parenteral compositions may be formulated in dosage-unit form for ease of administration and uniformity of dosage. Dosage-unit form, as used herein, refers to physically discrete units suited as unitary dosages for the subjects to be treated, each unit containing a predetermined quantity of active material calculated to produce the desired therapeutic effect in association with the required pharmaceutical carrier. The specification for the dosage-unit forms of the present application can be chosen based upon: (a) the unique characteristics of the active material and the particular therapeutic effect to be achieved, and (b) the limitations inherent in the art of compounding such an active material for the treatment of conditions in living subjects having a condition in which bodily health is impaired as described herein.

### D. Artificial Exon-Junction Complex

Another aspect of the present application relates to an artificial exon-junction complex comprising the fusion protein and the guide RNA of the present application. In some embodiments, the fusion protein comprises an RNA binding domain and an exon-junction complex (EJC) mimicking domain, and the guide RNA comprises a nucleotide sequence complementary to a target sequence in a target mRNA that contains a NMD-insensitive PTC.

### III. Method of Treatment

Another aspect of the present application relates to a method for preventing, treating or ameliorating a symptom of, a disease or condition caused by translation of a mRNA with a NMD-insensitive PTC (herein after the “target mRNA”) using the fusion protein of and the guide RNA of the present application. The fusion protein and the guide RNA may be expressed in situ in a target cell with one or more expression vectors.

In some embodiments, the method comprises the steps of: introducing into cells expressing the target mRNA: (1) a fusion protein comprising an RNA binding domain and an EJC mimicking domain, and (2) a guide RNA that binds specifically to a target region in the target mRNA, wherein the fusion protein, the guide RNA, and the target mRNA form a complex that results in the degradation of the target mRNA by NMD. In some embodiments, the fusion protein and the guide RNA are introduced into the cells by one or more expression vectors that express the fusion protein and the guide RNA in situ in the cells.

In some embodiments, the method comprises the steps of: administering to a subject in need of such treatment, an effective amount of one or more expression vectors of the present application as described herein. In some embodiments, the method comprises the steps of administering to a subject in need of such treatment, an effective amount of an expression vector comprising: a first nucleotide sequence encoding a RNA-binding domain; a second nucleotide sequence encoding an exon-junction complex (EJC) mimicking domain derived from one or more components of the EJC; and a regulatory sequence operably linked to at least one of the first and the second nucleotide sequence.

In some embodiments of the method, the first nucleotide sequence encodes a complete Cas13b nuclease or a RNA binding region thereof or a variant thereof.

In some embodiments of the method, the first nucleotide sequence encodes a complete protein region or the RNA-binding region/sequence of a Cas13d nuclease or a variant thereof.

In some embodiments of the method, the first nucleotide sequence encodes a complete protein region or the RNA-binding domain/sequence of a catalytically inactive Cas13d (dCas13d).

In some embodiments of the method, the catalytically inactive Cas nuclease is dRfxCas13d (SEQ ID. NO: 2), which harbors four positively charges amino acid substitutions (R295A, H300A, R849A and H854A).

In some embodiments of the method, the second nucleotide sequence encodes an EJC-mimicking domain comprising the complete protein region or a partial region/sequence of one or more EJC proteins.

In some embodiments of the method, the second nucleotide sequence encodes the EJC-mimicking domain comprising the complete protein region or a partial region/sequence of one or more of eIF4A3, Y14/RBM8A and MAGOH. In some embodiments, EJC-mimicking domain comprises the complete protein region or a partial region/sequence of one or more EJC proteins selected from the group consisting of elF4A3 (SEQ ID. NO: 12), Y14/RBM8A (SEQ ID. NO: 13), MAGOH (SEQ ID. NO: 14), RNPS1 (SEQ ID. NO: 27), SRSF1 (SEQ ID. NO: 28), UPF1 (SEQ ID. NO: 15), UPF2 (SEQ ID. NO: 16) and UPF3X/3B (SEQ ID. NO: 17).

In some embodiments of the method, the second nucleotide sequence encodes the EJC-mimicking domain comprising the complete protein region or a partial region of one or more EJC proteins selected from the group consisting of elF4A3 (SEQ ID. NO: 12), Y14/RBM8A (SEQ ID. NO: 13), MAGOH (SEQ ID. NO: 14), RNPS1 (SEQ ID. NO: 27), SRSF1 (SEQ ID. NO: 28), UPF1 (SEQ ID. NO: 15), UPF2 (SEQ ID. NO: 16) and UPF3X/3B (SEQ ID. NO: 17).

In some embodiments of the method, the second nucleotide sequence encodes the EJC-mimicking domain comprising the complete protein region or a partial region of one or more of eIF4A3 (SEQ ID. NO: 12), Y14/RBM8A (SEQ ID. NO: 13) and MAGOH (SEQ ID. NO: 14).

In some embodiments of the method, the expression vector further comprises a nucleotide sequence encoding a nuclear export signal. In some embodiments, the nuclear exporting signal comprises an amino acid sequence of SEQ ID NO: 18 or 19.

In some embodiments of the method, the method further comprises administering to the subject a guide RNA as described herein. In some embodiments, the guide RNA comprises a sequence complementary to a target sequence in a NMD-insensitive PTC-containing mRNA, wherein the 5′ end of the target sequence is located at least 10 nucleotides downstream from the NMD-insensitive PTC in the mRNA.

In some embodiments of the method, the guide RNA can be administered in combination with the expression vector that expresses the RNA binding domain and the EJC mimicking domain. In some embodiments of the method, the guide RNA is expressed under the same expression vector that expresses the RNA binding domain and the EJC-mimicking domain.

In some embodiments of the method, the guide RNA is expressed in a separate expression vector from the expression vector that expresses the RNA binding domain and the EJC-mimicking domain.

In some embodiments of the method, the method comprises the steps of administering to a subject in need of such treatment, an effective amount of a pharmaceutical composition comprising one or more expression vectors of the present application as described herein.

Yet in another aspect, the present application provides a method of inducing mRNA decay of a target mRNA comprising a premature termination codon (PTC) that is insensitive to the nonsense-mediated mRNA decay (NMD) process. In some embodiments, the method comprises the steps of introducing into cells expressing the target mRNA: (a) an expression vector comprising: (1) a first nucleotide sequence encoding a RNA binding domain, (2) a second nucleotide sequence encoding an exon-junction complex (EJC) mimicking domain derived from one or more components of the EJC, and (3) a regulatory sequence operably linked to at least one of the first and the second nucleotide sequence; and (b) a guide RNA that binds specifically to a target region in the target mRNA, wherein the expression vector expresses a fusion protein comprising a RNA binding domain and the EJC mimicking domain in the cells.

In some embodiments of the method, the first nucleotide sequence encodes a complete Cas13b nuclease or a RNA binding region thereof or a variant thereof.

In some embodiments of the method, the first nucleotide sequence encodes a complete protein region or the RNA binding region/sequence of a Cas13d nuclease or a variant thereof.

In some embodiments of the method, the first nucleotide sequence encodes a complete protein region or the RNA binding domain/sequence of a catalytically inactive Cas13d (dCas13d).

In some embodiments of the method, the catalytically inactive Cas nuclease is dRfxCas13d (SEQ ID. NO: 2), which harbors four positively charges amino acid substitutions (R295A, H300A, R849A and H854A).

In some embodiments of the method, the second nucleotide sequence encodes an EJC mimicking domain comprising the complete or a partial region/sequence of one or more EJC proteins.

In some embodiments of the method, the second nucleotide sequence encodes the EJC mimicking domain comprising the complete protein region or a partial region/sequence of one or more of eIF4A3, Y14/RBM8A and MAGOH. In some embodiments, EJC mimicking domain comprises the complete protein region or a partial protein region/sequence of one or more EJC proteins selected from the group consisting of elF4A3 (SEQ ID. NO: 12), Y14/RBM8A (SEQ ID. NO: 13), MAGOH (SEQ ID. NO: 14), RNPS1 (SEQ ID. NO: 27), SRSF1 (SEQ ID. NO: 28), UPF1 (SEQ ID. NO: 15), UPF2 (SEQ ID. NO: 16) and UPF3X/3B (SEQ ID. NO: 17).

In some embodiments of the method, the second nucleotide sequence encodes the EJC-mimicking domain comprising the complete protein region or a partial region of one or more EJC proteins selected from the group consisting of elF4A3 (SEQ ID. NO: 12), Y14/RBM8A (SEQ ID. NO: 13), MAGOH (SEQ ID. NO: 14), RNPS1 (SEQ ID. NO: 27), SRSF1 (SEQ ID. NO: 28), UPF1 (SEQ ID. NO: 15), UPF2 (SEQ ID. NO: 16) and UPF3X/3B (SEQ ID. NO: 17).

In some embodiments of the method, the second nucleotide sequence encodes the EJC-mimicking domain comprising the complete protein region or a partial region of one or more of eIF4A3 (SEQ ID. NO: 12), Y14/RBM8A (SEQ ID. NO: 13) and MAGOH (SEQ ID. NO: 14).

In some embodiments of the method, the expression vector further comprises a nucleotide sequence encoding a nuclear export signal. In some embodiments, the nuclear exporting signal comprises an amino acid sequence of SEQ ID NO: 18 or 19.

In some embodiments of the method, the method further comprises administering to the subject a guide RNA. In some embodiments, the guide RNA comprises a sequence complementary to a target sequence in a NMD-insensitive PTC-containing mRNA, wherein the 5′ end of the target sequence is located at least 10 nucleotides downstream from the NMD-insensitive PTC in the mRNA.

In some embodiments of the method, the guide RNA can be administered in combination with the expression vector that expresses the RNA-binding domain and the EJC mimicking domain. In some embodiments, the guide RNA is expressed under the same expression vector that expresses the RNA binding domain and the EJC mimicking domain.

In some embodiments of the method, the guide RNA is expressed in a separate expression vector from the expression vector that expresses the RNA-binding domain and the EJC-mimicking domain.

### A. Target Diseases and Conditions

Examples of disease or condition caused by translation of a mRNA with a NMD-insensitive PTC include, but are not limited to, diseases and conditions listed in Table

TABLE 1
Genetic Conditions in which a PTC can modulate disease phenotype

| Disease | Gene Name | Phenotype |
|---|---|---|
| Thalassemia-beta | β-Globin (HBB) | NMD-sensitive PTC: recessively |
| Thalassemia-beta, dominant | | inherited β-thalassaemia major; |
| inclusion-body | | heterozygotes healthy |
| | | NMD-insensitive PTC: |
| | | dominantly inherited β- |
| | | thalassaemia intermedia |
| Retinitis Pigmentosa | Rhodopsin (RHO) | NMD-sensitive PTC: recessively |
| Retinitis punctata albescens | | inherited blindness; heterozygotes |
| | | have abnormalities on retinogram, |
| | | but no clinical disease |
| | | NMD-insensitive PTC: |
| | | dominantly inherited blindness |
| Cone-rod retinal dystrophy | Cone-rod homeobox containing | NMD-sensitive PTC: no |
| Leber congenital amaurosis 7 | gene (CRX) | homozygotes to date; |
| | | heterozygotes healthy |
| | | NMD-insensitive PTC: |
| | | dominantly inherited Leber |
| | | congenital amaurosis |
| Aniridia | Paired box gene 6 (PAX6) | NMD-sensitive PTC: aniridia, |
| Anterior Segment Dysgenesis 5 | | congenital absence of the iris |
| Foveal Hypoplasia 1 | | NMD-insensitive PTC: |
| Keratitis | | dominant-negative protein is |
| | | predicted to show severe |
| | | phenotype |
| PCWH syndrome | SRY-box 10 (SOX10) | NMD-sensitive PTC: |
| Waardenburg syndrome | | haploinsufficiency leading to |
| | | congenital neurosensory deafness |
| | | and colonic agangliosis |
| | | NMD-insensitive PTC: |
| | | dominantly inherited neural |
| | | developmental defect including |
| | | neurosensory deafness, colonic |
| | | agangliosis, peripheral |
| | | neuropathy and central |
| | | dysmyelinating leukodystrophy |
| Osteogenesis imperfecta | Collagen type I, α1 (COL1A1) | NMD-sensitive PTC: dominantly |
| | | inherited, osteogenesis imperfecta |
| | | (OI) type I (mild form) |
| | | NMD-insensitive PTC: OI type |
| | | II-IV (severe form) |
| Spinal muscular atrophy | Survival motor neuron gene | NMD-sensitive PTC: spinal |
| | (SMN1) | muscular atrophy (SMA) type III |
| | | (mild form) |
| | | NMD-insensitive PTC: spinal |
| | | muscular atrophy (SMA) type I |
| | | (severe form) |
| Myotonia congenita | Chloride channel 1, skeletal | NMD-sensitive PTC: recessively |
| | muscle (CLCN1) | inherited Becker disease |
| | | NMD-insensitive PTC: |
| | | dominantly inherited Thomsen |
| | | disease (muscular disorder |
| | | characterized by muscle stiffness |
| | | and an inability of the muscle to |
| | | relax) |
| Charcot-Marie-Tooth disease | Myelin protein zero (MPN) | NMD-sensitive PTC: |
| Dejerine-Sottas disease | | haploinsufficiency, Charcot- |
| Hypomyelinating neuropathy, | | Marie-Tooth disease (neuropathy |
| congenital, 2 | | with loss of muscle tissue and |
| | | touch sensation) |
| Roussy-Levy syndrome | | NMD-insensitive PTC: |
| | | dominant-negative or gain-of- |
| | | function, congenital |
| | | hypomyelinating neuropathy |
| Ataxia-telangiectasia | Ataxia-telangiectsia mutated | NMD-sensitive PTC: mild form |
| | gene (ATM) | of ataxia |
| | | NMD-insensitive PTC: severe |
| | | form with short survival time |
| Craniosynostosis | Zinc finger protein of | NMD-sensitive PTC: Dandy- |
| Dandy-Walker malformation | cerebellum 1 (ZIC1) | Walker malformation of the |
| | | cerebellum |
| | | NMD-insensitive PTC: severe |
| | | craniosynostosis, specifically |
| | | involving the coronal sutures, |
| | | and variable learning disability |
| Saethre-Chotzen Syndrome | Twist basic helix-loop helix | NMD-insensitive PTC: cone- |
| Robinow-Sorauf Syndrome | transcription factor 1 | shaped head, asymmetrical |
| | (TWIST1) | face, hand and foot |
| | | malformation, and mental |
| | | retardation |
| Craniofrontonasal syndrome | Ephrin-B1 (EFNB1) | NMD-insensitive PTC: |
| | | Craniofrontonasal dysplasia |
| | | including severe hypertelorism, |
| | | depressed nasal bridge and bifid |
| | | nasal tip, frontal bossing, |
| | | coronal suture synostosis, corpus |
| | | callosum agenesis, and |
| | | occasionally cleft lip or palate |
| Frontonasal dysplasia 2 | ALX homeobox 4 (ALX4) | NMD-sensitive PTC: recessively |
| Parietal foramina 2 | | inherited frontonasal dysplasia |
| | | NMD-insensitive PTC: |
| | | dominantly inherited parietal |
| | | foramina, including |
| | | hypertelorism, lack of formation |
| | | of the nasal tip, and broad and |
| | | elongated columella |
| Townes-Brocks syndrome | SAL-like 1 (SALL1) | NMD-sensitive PTC: milder |
| | | Townes-Brocks syndrome |
| | | NMD-insensitive PTC: |
| | | dominantly inherited |
| | | imperforate anus, dysplastic |
| | | ears, and thumb malformations |
| Malan overgrowth syndrome | Nuclear factor I/X (NFIX) | NMD-sensitive PTC: advanced |
| Marshall-Smith syndrome | | bone age, macrocephaly, |
| | | developmental delay, scoliosis, |
| | | and unusual facies |
| | | NMD-insensitive PTC: severe |
| | | malformation syndrome |
| | | characterized by failure to |
| | | thrive, respiratory insufficiency, |
| | | accelerated osseous maturation, |
| | | kyphoscoliosis, osteopenia, and |
| | | unusual facies. |
| Immunodeficiency 27A | Interferon-γreceptor 1 | NMD-sensitive PTC: recessively |
| Immunodeficiency 27B | (IFNGR1) | inherited susceptibility to |
| | | mycobacterial infection |
| | | NMD-insensitive PTC: |
| | | dominantly inherited |
| | | susceptibility to mycobacterial |
| | | infection |
| Robinow syndrome | Receptor tyrosine kinase-like | NMD-sensitive PTC: recessively |
| Brachydactyly, type B1 | orphan receptor 2 (ROR2) | inherited Robinow syndrome |
| | | (characteristic facial features, |
| | | orodental abnormalities, and |
| | | hypoplastic genitalia.); |
| | | heterozygotes healthy |
| | | NMD-insensitive PTC: most |
| | | severe type of human |
| | | brachydactyly, and shows high |
| | | penetrance and variable |
| | | expressivity. Hypoplastic or |
| | | absent distal phalanges and nails |
| | | of digits 2 through 5 in the hands |
| | | and feetdominantly inherited |
| | | brachydactyly type B1 |
| von Willebrand disease | Von Willebrand factor (VWF) | NMD-sensitive PTC: recessively |
| | | inherited von Willebrand disease |
| | | (defect in platelet aggregation, |
| | | prolonged bleeding time); |
| | | heterozygous healthy |
| | | NMD-insensitive PTC: |
| | | mucocutaneous bleeding, such as |
| | | epistaxis and menorrhagia, and |
| | | prolonged bleeding after surgery |
| | | or trauma. |
| Factor X deficiency | Coagulation factor X (F10) | NMD-sensitive PTC: recessively |
| | | inherited bleeding tendency; |
| | | heterozygous healthy |
| | | NMD-insensitive PTC: |
| | | dominantly inherited bleeding |
| | | tendency |
| Supravalvar aortic stenosis | Elastin (ESN) | NMD-sensitive PTC: |
| Cutis laxa | | haploinsufficiency; obstructive |
| | | arterial disease, supravalvular |
| | | aortic stenosis |
| | | NMD-insensitive PTC: severe |
| | | congenital pulmonary disease |
| | | and supravalvular pulmonary |
| | | artery stenosis |
| Ectodermal dysplasia 10A | Ectodysplasin A receptor | NMD-sensitive PTC: recessively |
| Ectodermal dysplasia 10B | (EDAR) | inherited ectodermal dysplasia; |
| | | abnormal morphogenesis of teeth, |
| | | hair, and eccrine sweat glands |
| | | NMD-insensitive PTC: |
| | | dominantly inherited ectodermal |
| | | dysplasia |
| Pseudohypoaldosteronism, type I | Sodium channel epithelial 1 | NMD-sensitive PTC: recessive |
| Liddle syndrome 1 | subunit beta (SCNN1B) | inherited |
| | | pseudohypoaldosteronism type I |
| | | NMD-insensitive PTC: early- |
| | | onset salt-sensitive hypertension, |
| | | hypokalemia, metabolic |
| | | alkalosis, and suppression of |
| | | plasma renin activity and |
| | | aldosterone secretion |
| Breast-ovarian cancer, familial | Breast cancer 1 (BRCA1) | NMD-sensitive PTC: increased |
| | Breast cancer 2 (BRCA2) | cancer susceptibility |
| | | NMD-insensitive PTC: increased |
| | | chemoresistance, decreased |
| | | apoptosis, increased |
| | | tumorigenicity and decreased |
| | | survival time |
| Wilms tumor, type 1 | WT1 transcription factor | NMD-sensitive PTC: increased |
| Denys-Drash syndrome | (WT1) | cancer susceptibility |
| | | NMD-insensitive PTC: |
| | | dominantly inherited Wilms |
| | | tumor, pseudohermaphroditism, |
| | | hypertension, and degenerative |
| | | renal disease |
| Adenomatous polyposis coli | Adenomatous polyposis coli | NMD-sensitive PTC: increased |
| Adenoma, periampullary, somatic | (APC) | cancer susceptibility |
| Brain tumor-polyposis syndrome 2 | | NMD-insensitive PTC: promote |
| Colorectal cancer, somatic | | the Wnt/β-catenin signaling |
| Desmoid disease, hereditary | | pathway, affecting cancer |
| Gardner syndrome | | progression and severity |
| Adrenocortical carcinoma | Tumor protein p53 (TP53) | NMD-sensitive PTC: increased |
| Breast cancer, somatic | | cancer susceptibility |
| Bone marrow failure syndrome 5 | | NMD-insensitive PTC: promote |
| Colorectal cancer | | cancer cell proliferation, |
| Hepatocellular carcinoma, somatic | | survival, and metastasis |
| Li-Fraumeni syndrome | | |
| Pancreatic cancer, somatic | | |
| Rett syndrome, congenital variant | Forkhead box G1 (FOXG1) | NMD-insensitive PTC: postnatal |
| | | microcephaly, seizures and |
| | | severe mental retardation |

### B. Guide RNA

The guide RNA is designed to target the NMD-insensitive PTC-containing mRNA that causing the disease or condition. Specifically, the guide RNA comprises a nucleotide sequence that is complementary to a target sequence in the NMD-insensitive PTC-containing mRNA. In some embodiments, the 5′ end of the target sequence is at least 10, 15, 20, 25 or 30 nucleotides downstream of the NMD-insensitive PTC and the target sequence is within the normal protein-coding sequence of the normal mRNA translation reading frame. In some embodiments, the 5′ end of the target sequence is at least 20-24 nucleotides downstream of the NMD-insensitive PTC.

In some embodiments, the target sequence comprises at least 21 nucleotides to maintain binding specificity of the guide RNA. In some embodiments, the guide RNA comprises a hairpin structure. In some embodiments, the hairpin structure is 30-36 nt in length and contains an 8-10-nucleotide stem with A/U-rich loop. An exemplary guide RNA sequence is shown in SEQ ID NO:21. The guide RNA is specifically designed for the treatment of colorectal cancer using the method of the present application.

### C. Route of Administration

The fusion protein and guide RNA of the present application may be introduced into a target cell or administered into a subject using methods well-known in the art. In some embodiments, the fusion protein and guide RNA of the present application introduced into a target cell in the form of one or more expression vectors. The one or more expression vectors express the fusion protein and the guide RNA in the target cell, which then leads to the degradation of the NMD-insensitive PTC-containing mRNA in the target cell.

In some embodiments, the one or more expression vectors are administered to a subject by injection or infusion, such as intravenous, intramyocardial, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrastemal injection or infusion.

### EXAMPLES

### Example 1: An RNA-Programed NMD Activation (RP-NMDA) System

Bacteriophage protein-tethering systems require grafting exogenous RNA-binding sequences to target mRNAs, which is a significant drawback to applying this system to disease therapy. In contrast, the class 2 type VI CRISPR and Cas13 (CRISPR-Cas13) system is a robust and programmable molecular tool for RNA. To target NMD-insensitive PTC-containing transcripts, CRISPR-Cas13 technology is used to establish a new methodology of selective RNA degradation. The Cas13 family is the only family of class 2 Cas enzymes that exclusively cleave single-stranded RNAs using two higher eukaryotes and prokaryote nucleotide (HEPN)-binding domains.

Ruminococcus flavefaciens Cas13d (also known as CasRx) is selected because Cas13d (1) is used as it has better on-target RNA specificity compared to other Cas13s and shRNAs, (2) is among the smallest (˜930 aa) class 2 Cas enzyme reported to date, and (3) does not require the protospacer flanking sequence (PFS) located at the 3′-end of the spacer sequence, which overcomes a restriction in designing guide RNA sequences.

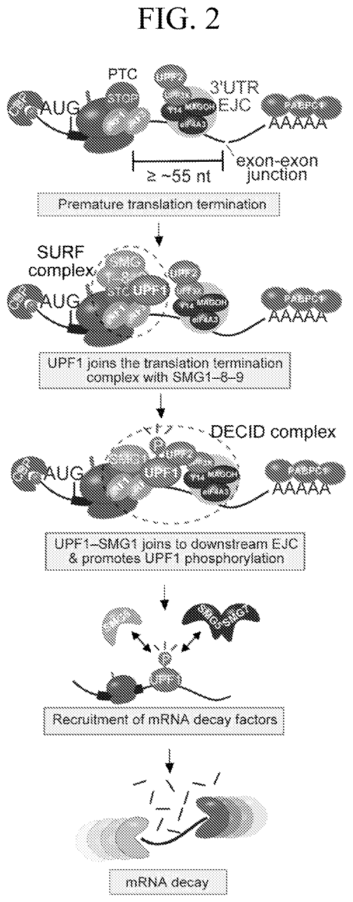

To selectively digest PTC-containing mRNAs, the RNA programmable RP-NMDA system is established by mimicking the 3′UTR EJC-dependent NMD mechanism (FIG. 2). A catalytically inactive Cas13d (dCas13d) is used in this example. dCas13d harbors four positively charged amino acid substitutions (R295A, H300A, R849A, and H854A) to inactivate Cas13d HEPN nuclease activity. By doing so, digesting off-target transcripts derived from the normal allele can be avoided.

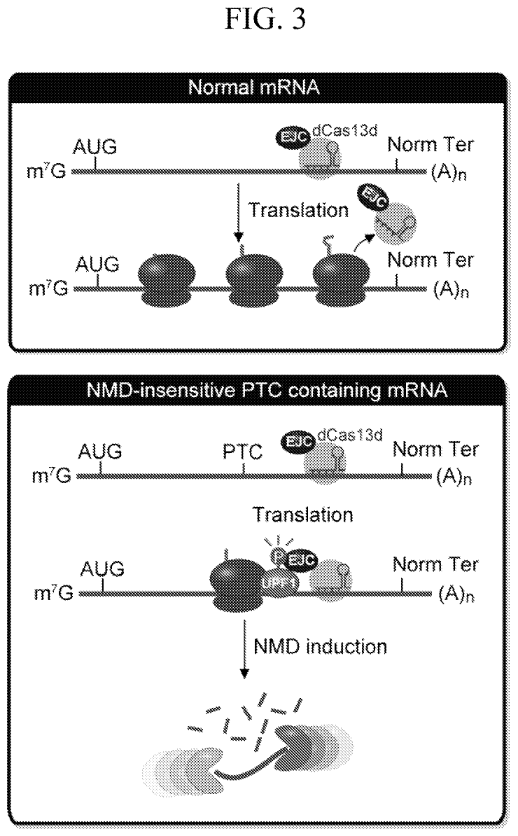

Furthermore, dCas13d fused to an EJC constituent (eIF4A3, Y14/RBM8A, or MAGOH) is generated to activate the NMD pathway. A guide RNA is designed that hybridizes to a position situated between the NMD-insensitive PTC and the normal termination codon according to the “˜55-nt rule” of NMD. This position is preferably located at least 20-24 nt downstream of any PTCs, since NMD sensitivity requires a PTC to be >20-24 nt from a downstream EJC at the 3′-end to efficiently activate NMD. Since ribosomes displace any proteins deposited on mRNA coding regions, it is expected that the EJC-dCas13d on the coding region of normal mRNAs are efficiently removed by translocating ribosomes during the first round of translation (upper panel FIG. 3). Whereas, EJC-dCas13d positioned downstream of the PTC on NMD-insensitive mRNAs remains, since the translating ribosome does not proceed past the PTC and remove it, inducing NMD (lower panel in FIG. 3).

Since Cas13d does not require PFS, this RP-NMDA system is applicable to any positions on disease-causing NMD-insensitive transcripts that satisfy the rules of NMD engagement. The fusion protein are expressed with mammalian expression vectors encoding dCas13d (pXR002/EF1a-dCasRx-2A-EGFP; Addgene), guide RNA (pXR003/CasRx gRNA cloning backbone; Addgene), and EJC constituent (pcDNA3-HA-eIF4A3; pcDNA-FLAG-Y14/RBM8A and pcDNA-FLAG-MAGOH).

### Example 2: Effect of the RP-NMDA System

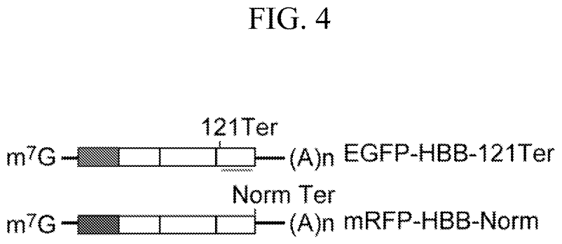

To evaluate the effect of RP-NMDA, the reporter construct (pEGFP-HBB-121 Ter) is generated by harboring NMD-insensitive beta-thalassemia gene mutations at position 121 of the amino acid sequence (121 Ter) from the beta-globin (HBB) start codon using pEGFP-N1 (Clontech) and pFLAG-CMV2-HBB (FIG. 4). Due to the lack of a 3′UTR EJC, mRNAs bearing 121 Ter in the last exon of the HBB gene are insensitive to NMD, resulting in a dominantly inherited and severe form of beta-thalassemia where truncated proteins accumulate and form insoluble inclusion bodies.

As a negative control, the reporter construct (pmRFP-HBB-Norm) is generated harboring normal HBB mRNA using pmRFP-N1 (Clontech) and pFLAG-CMV2-HBB (FIG. 4). Using the human cervical cancer cell line (HeLa CCL-2; ATCC), these reporter plasmids are introduced into HeLa cells using Lipofectamine 2000 (Thermo Fisher Scientific) and establish a stable cell line expressing both reporter constructs (pEGFP-HBB-121Ter and pmRFP-HBB-Norm) selected by protein fluorescence and Geneticin (G418) treatment.

By introducing EJC-dCas13d and gRNA into HeLa cells stably expressing HBB mRNA reporters, mRNA abundance is measured by reverse transcription coupled to quantitative PCR (RT-qPCR) using QuantStudio 5 Real-Time PCR System (Thermo Fisher Scientific) and protein abundance by western blotting and fluorescent assay using a microplate reader (SpectraMax M4, Molecular Devices). Inclusion-body formation are monitored using a fluorescent microscope (EVOS FL Cell Imaging System, Thermo Fisher Scientific) and FV-1000 Confocal Laser Microscope (OLYMPUS).

When most effective EJC-dCas13d variant is found, deletion variants of the EJC constituent are generated to reduce the size of the effective protein. The size reduction may be important for future RP-NMDA applications since smaller sizes are necessary for packaging in low-capacity virus vectors that minimize immunogenicity in gene therapy treatments.

### Example 3: Physiological Effect of RNA-Programmed NMD Induction on Human Colorectal Cancer Cell Lines

Colorectal cancer is the third leading cause of cancer-related deaths in the United States, with a lifetime risk of about 1 in 23 (4.3%) for men and 1 in 25 (4%) for women. Mutations in the tumor suppressor gene Adenomatous Polyposis Coli (APC) are one of the most frequently observed gene alterations in colorectal cancer cells. Somatic mutations in the APC gene associate with sporadic colorectal carcinomas (up to 80%), and germline APC mutations associate with the autosomal dominant familial adenomatous polyposis. Notably, more than 85% of APC mutations generate truncated APC proteins in colorectal cancer cells, most of which (˜60%) occur within the region known as the mutation cluster region (MCR). Truncated APC proteins promote the Wnt/β-catenin signaling pathway, affecting cancer progression and severity. However, the underlying molecular mechanism of how truncated proteins disrupt normal cellular function remains largely unknown.

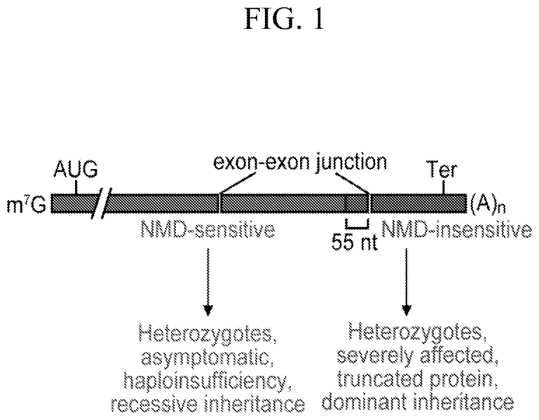

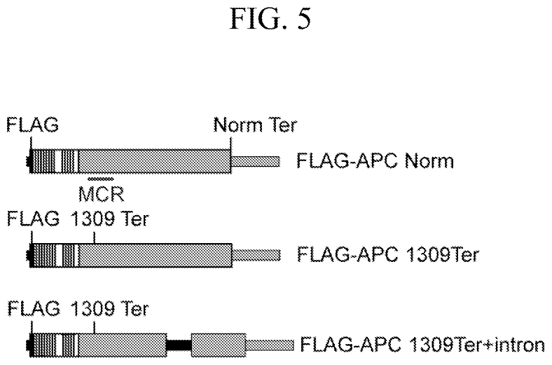

Since most somatic mutations in the APC gene are exclusively observed in the last exon, which occupies ˜80% of the APC gene, the truncated APC protein production in colorectal cancer is most likely a result of the insensitivity to NMD (FIG. 1). To prove the NMD-insensitivity of the PTCs in APC mRNA, a normal APC-expressing reporter construct (pCMV-Neo-Bam APC; Addgene) and mutated APC-expressing reporter construct containing 1309 PTC (pCMV-Neo-Bam APC 1-1309; Addgene) are obtained. Then, the FLAG-tag sequence is inserted to generate pCMV-Neo-FLAG-APC and pCMV-Neo-FLAG-APC-1309Ter for protein analysis. Using pCMV-Neo-FLAG-APC-1309Ter, The HBB intron 1 (130 bp) is further inserted downstream of APC 1309Ter to generate pCMV-Neo-FLAG-APC-1309Ter+intron to test for NMD induction on this construct (FIG. 5).

Generated mammalian-expression plasmids and the loading control plasmid pcDNA-EGFP are transfected into HeLa cells using Lipofectamine 2000. In parallel, either a negative control siRNA (Silencer Negative Control #1 siRNA; Thermo Fisher Scientific) or UPF1 siRNA is introduced into pCMV-Neo-FLAG-APC-1309Ter+intron expressing HeLa cells using Lipofectamine RNAiMAX (Thermo Fisher Scientific) to test any observed reporter mRNA degradation for dependence on NMD.

Two days later, cells are harvested to measure the mRNA abundance by RT-qPCR and protein abundance by semi-quantitative western blotting using anti-FLAG HRP conjugated antibody (Sigma Aldrich). If mRNA abundance reduction is observed with APC 1309Ter+intron mRNAs but not APC 1309Ter mRNAs, and UPF1 downregulation restores the mRNA reduction of APC 1309Ter+intron mRNAs, this is proof of concept for the RP-NMDA application. Then, the most effective EJC-dCas13d described herein and guide RNAs are applied to HeLa cells expressing APC Norm or APC 1309Ter mRNAs to evaluate the efficacy of NMD induction and target-specificity for PTC-containing mRNAs.

Next, the RP-NMDA approach are applied to colorectal cancer cell lines (SW480, CaCo2, DLD-1, Lovo, SW948, and HT-29; ATCC) by selectively designing guide RNAs to target transcripts encoding APC truncations in these cell types. CCD 841 CON cells (ATCC) are used as a normal control cell line. RP-NMDA construct (pXR002 and pXR003) are transfected into the colorectal cancer cell lines or normal control cell lines using the NEON Transfection System (Thermo Fisher Scientific). Protein abundance of APC and factors involved in Wnt/β-catenin signaling are measured by western blotting using specific antibodies: anti-APC (Ali 12-28; Abcam), anti-β-catenin (E-5; Santa Cruz Biotechnologies), anti-Axin2 (76G6; Cell Signaling), and anti-Lgr5 (OTI2A2; Thermo Fisher Scientific). RT-PCR measures APC and another Wnt/β-catenin signaling pathway-related mRNA abundances, and 3-(4,5-dimethylthiazol-2-yl)-2,5-diphenyltetrazolium bromide (MTT) assay are performed for evaluating cell viability and proliferation using Vybrant MTT Cell Proliferation Assay Kit (Thermo Fisher Scientific). Cell migration and invasion are measured using QCM Tumor Cell Trans-Endothelial Migration Assay Kit (Millipore). Wnt/β-catenin signaling activity is assessed using TCF/LEF Reporter Kit (BPS Bioscience). Furthermore, the effect of RP-NMDA are thoroughly evaluated by transcriptome-wide RNA sequencing comparing colorectal cancer cells with normal control cells.

To avoid potential artifacts and off-target effects, three independent primer sets or siRNA sequences are used for each target. For statistics, power analysis (power=0.80 and α=0.05 for two-sided statistical tests) determines the sample size needed for a reliable statistical judgment. Statistical significance are calculated using a two-sided unpaired t-test, two-sided Wilcoxon rank-sum test, one-way ANOVA Dunnett's multiple comparison test, Pearson's correlation coefficient test, or Fisher's exact test using R version 4.0.5. All R scripts used for data processing and statistical analysis are described in publications or provided upon request.

### Example 4: NMD Reporter System Harboring NMD-Sensitive or NMD-Insensitive PTCs

The study determines if NMD can be inducible for the disease-causing NMD-insensitive PTCs. As a disease model, the study initially focuses on the red blood cell disorder beta-thalassemia. The main goal is to (i) determine if NMD can be inducible on the NMD-insensitive PTC-containing mRNAs and (ii) to define which NMD factors effectively induce NMD for NMD-insensitive PTCs. The study performs the following experiments.

First, the study utilizes the bacteriophage MS2 coat protein system to prove the concept. As an NMD reporter, the study uses the human beta-globin (HBB) gene harboring NMD-insensitive nonsense mutations at codon 121 (E121X) and codon 127 (Q127X). Those mutations cause dominantly inherited inclusion body beta-thalassemia characterized by moderately severe anemia, jaundice, and splenomegaly.

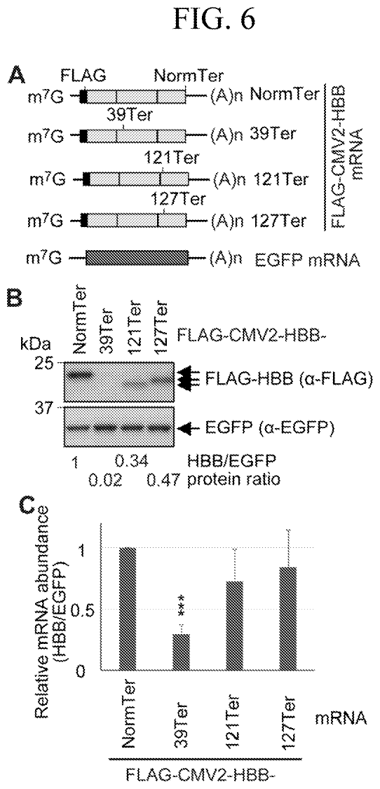

The study generated an NMD reporter plasmid expressing FLAG-tag fused HBB mRNAs (FLAG-HBB) containing either normal termination codon (NormTer), NMD-sensitive PTC (39Ter), or NMD-insensitive PTCs (E121X: 121Ter or Q127X: 127Ter) together with transfection control EGFP mRNAs (FIG. 6, panel A). The study transfected these NMD reporter plasmids into human embryonic kidney (HEK) 293T cells. Western blotting analysis using HEK293T cell lysates indicates that 34% or 47% of the wild-type level of truncated HBB proteins were expressed in FLAG-HBB 121Ter or FLAG-HBB 127Ter, respectively (FIG. 6, panel B). Quantitative reverse-transcribed PCR (RT-qPCR) analysis indicates that FLAG-HBB 121Ter mRNAs and FLAG-HBB 127Ter mRNAs are insensitive for NMD (FIG. 6, panel C).

The study introduces NMD reporter plasmids with multiple MS2 coat protein binding sequences to generate pFLAG-CMV2-HBB-6MS2bs NormTer, pFLAG-CMV2-HBB-6MS2bs 39Ter, pFLAG-CMV2-HBB-6MS2bs 121Ter, and pFLAG-CMV2-HBB-6MS2bs 127Ter into HEK293T cells. For the selection of the most effective NMD effector, the study tests several potent NMD activators, such as UPF1, eIF4A3, RBM8A/Y14, MAGOH, or SRSF1. The study tethers one of the potent NMD activator downstream of the NMD-insensitive PTCs on HBB mRNAs using a bacteriophage MS2 coat protein and MS2 RNA binding system. The study already generated plasmids expressing MS2 coat protein fused NMD effectors (pcNMS2-UPF1, pcNMS2-eIF4A3, pcNMS2-RBM8A/Y14, pcNMS2-MAGOH, and pcNMS2-SRSF1).

Using these plasmid constructs, the study introduces an NMD reporter (pFLAG-CMV2-HBB-6MS2bs NormTer, pFLAG-CMV2-HBB-6MS2bs 121Ter, or pFLAG-CMV2-HBB-6MS2bs 127Ter), an NMD effector plasmid (pcNMS2, pcNMS2-UPF1, pcNMS2-eIF4A3, pcNMS2-RBM8A/Y14, pcNMS2-MAGOH, pcNMS2-SRSF1), and a transfection control plasmid (pcDNA-EGFP) into HEK293T cells using the TransIT-X2 Dynamic Delivery system (Mirus Bio). After two days of incubation (48 hours), cells are collected to measure the HBB protein abundance by western blotting using monoclonal anti-FLAG M2 antibody (Sigma-Aldrich) and HBB mRNA abundance by RT-qPCR using HBB-specific primers. As a transfection control, EGFP proteins and mRNAs are analyzed. HBB inclusion bodies, which are HBB protein aggregates, are measured by immunofluorescence and confocal laser scanning microscope (OLYMPUS FV-1000). HBB protein and mRNAs are downregulated, and the number and the size of HBB inclusion bodies are reduced by the tethering of NMD activators; the study concludes that NMD can be inducible for disease-causing NMD-insensitive PTCs. By comparing several NMD activators, the study selects the most effective NMD activator for applying the downstream analysis.

### Example 5: A Method to Trigger NMD of Dominant-Negative Transcripts

Bacteriophage MS2 coat protein and MS2 RNA binding system require grafting exogenous RNA-binding sequences to target mRNAs, which is a significant drawback to applying this system to disease therapy. In contrast, the class 2 type VI CRISPR and Cas13 (CRISPR-Cas13) system is a robust and programmable molecular tool for RNA editing. To target NMD-insensitive PTC-containing transcripts, we first aim to use CRISPR-Cas13 technology to establish a new methodology of selective RNA degradation. The Cas13 family is the only family of class 2 Cas enzymes that exclusively cleave single-stranded RNAs using two higher eukaryotes and prokaryote nucleotide (HEPN)-binding domains. The study uses Ruminococcus flavefaciens Cas13d (also known as CasRx) because Cas13d (1) has better on-target RNA specificity compared to other Cas13s and shRNAs, (2) is among the smallest (˜930 aa) class 2 Cas enzyme reported to date, and (3) does not require the protospacer flanking sequence (PFS) located at the 3′-end of the spacer sequence, which overcomes a restriction in designing guide RNA sequences.

To selectively digest PTC-containing mRNAs, an RNA programmable RNA-Programmed NMD Activation (RP-NMDA) system is established by mimicking the 3′UTR EJC-dependent NMD mechanism (FIG. 2). Catalytically inactive Cas13d (dCas13d), which harbors four positively charged amino acid substitutions (R295A, H300A, R849A, and H854A), is used to inactivate Cas13d HEPN nuclease activity. The use of dCas13d avoids digesting transcripts derived from the normal allele. Furthermore, dCas13d is fused to an EJC constituent (eIF4A3, Y14/RBM8A, or MAGOH) or NMD activator (UPF1, RNPS1, or SRSF1) to activate the NMD pathway (FIG. 3). A guide RNA is designed to hybridize to a position situated between the NMD-insensitive PTC and the normal termination codon according to the “˜55-nt rule” of NMD. This position is located at least 20-24 nt downstream of any PTCs, since NMD sensitivity requires a PTC to be >20-24 nt from a downstream EJC at the 3′-end to efficiently activate NMD. Since ribosomes displace any proteins deposited on mRNA coding regions, the study expects that the EJC-dCas13d on the coding region of normal mRNAs are efficiently removed by translocating ribosomes during the first round of translation (upper panel in FIG. 3). Whereas, EJC-dCas13d positioned downstream of the PTC on NMD-insensitive mRNAs remain, since the translating ribosome does not proceed past the PTC and remove it, inducing NMD (lower panel in FIG. 3). Since Cas13d does not require PFS, this RP-NMDA system is theoretically applicable to any positions on disease-causing NMD-insensitive transcripts that satisfy the rules of NMD engagement.

To evaluate the effect of RP-NMDA, the study utilizes the reporter construct, pFLAG-CMV2-HBB NormTer, pFLAG-CMV2-HBB 39Ter, pFLAG-CMV2-HBB 121Ter, or pFLAG-CMV2-HBB 127Ter (FIG. 6, panel A). As a negative control, the study uses pmRFP reference plasmid. Using the HEK293T cells, the study introduces these reporter plasmids into HEK293T cells using TransIT-X2 Dynamic Delivery system (Mirus Bio).

By introducing dCas13d plasmid (pXR002-UPF1, eIF4A3, RBM8A/Y14, MAGOH, RNPS1, or SRSF1) and gRNA into HEK293T cells expressing HBB mRNA reporters, the study measures mRNA abundance by RT-qPCR using QuantStudio 5 Real-Time PCR System (Thermo Fisher Scientific) and protein abundance by western blotting and fluorescent assay using a microplate reader (SpectraMax M4, Molecular Devices). Inclusion body formation are monitored using a fluorescent microscope (EVOS FL Cell Imaging System, Thermo Fisher Scientific) and FV-1000 Confocal Laser Microscope (OLYMPUS). The study finds the most effective dCas13d plasmid, the study generates deletion variants of the NMD effector to reduce the size of the effective protein. The size reduction may be important for future RP-NMDA applications since smaller sizes are necessary for packaging in low-capacity virus vectors that minimize immunogenicity in gene therapy treatments.

LISTING OF SEQUENCES

| SEQ | | |
|---|---|---|
| ID | | |
| NO: | Description | Sequence |
| 1 | Cas13d | MIEKKKSFAKGMGVKSTLVSGSKVYMTTFAEGSDARLEKIVEGDSIRS |
| | (RfxCas13d) | VNEGEAFSAEMADKNAGYKIGNAKFSHPKGYAVVANNPLYTGPVQQDM |
| | | LGLKETLEKRYFGESADGNDNICIQVIHNILDIEKILAEYITNAAYAV |
| | | NNISGLDKDIIGFGKFSTVYTYDEFKDPEHHRAAFNNNDKLINAIKAQ |
| | | YDEFDNFLDNPRLGYFGQAFFSKEGRNYIINYGNECYDILALLSGLRH |
| | | WVVHNNEEESRISRTWLYNLDKNLDNEYISTLNYLYDRITNELTNSFS |
| | | KNSAANVNYIAETLGINPAEFAEQYFRFSIMKEQKNLGFNITKLREVM |
| | | LDRKDMSEIRKNHKVFDSIRTKVYTMMDFVIYRYYIEEDAKVAAANKS |
| | | LPDNEKSLSEKDIFVINLRGSFNDDQKDALYYDEANRIWRKLENIMHN |
| | | IKEFRGNKTREYKKKDAPRLPRILPAGRDVSAFSKLMYALTMELDGKE |
| | | INDLLTTLINKFDNIQSFLKVMPLIGVNAKFVEEYAFFKDSAKIADEL |
| | | RLIKSFARMGEPIADARRAMYIDAIRILGTNLSYDELKALADTFSLDE |
| | | NGNKLKKGKHGMRNFIINNVISNKRFHYLIRYGDPAHLHEIAKNEAVV |
| | | KFVLGRIADIQKKQGONGKNQIDRYYETCIGKDKGKSVSEKVDALTKI |
| | | ITGMNYDQFDKKRSVIEDTGRENAEREKFKKIISLYLTVIYHILKNIV |
| | | NINARYVIGFHCVERDAQLYKEKGYDINLKKLEEKGFSSVTKLCAGID |
| | | ETAPDKRKDVEKEMAERAKESIDSLESANPKLYANYIKYSDEKKAEEF |
| | | TROINREKAKTALNAYLRNTKWNVIIREDLLRIDNKTCTLFRNKAVHL |
| | | EVARYVHAYINDIAEVNSYFQLYHYIMQRIIMNERYEKSSGKVSEYFD |
| | | AVNDEKKYNDRLLKLLCVPFGYCIPRFKNLSIEALFDRNEAAKEDKEK |
| | | KKVSGNS |
| 2 | dCas13d | MIEKKKSFAKGMGVKSTLVSGSKVYMTTFAEGSDARLEKIVEGDSIRS |
| | (dRfxCas13d) | VNEGEAFSAEMADKNAGYKIGNAKFSHPKGYAVVANNPLYTGPVQQDM |
| | | LGLKETLEKRYFGESADGNDNICIQVIHNILDIEKILAEYITNAAYAV |
| | | NNISGLDKDIIGFGKFSTVYTYDEFKDPEHHRAAFNNNDKLINAIKAQ |
| | | YDEFDNFLDNPRLGYFGQAFFSKEGRNYIINYGNECYDILALLSGLAH |
| | | WVVANNEEESRISRTWLYNLDKNLDNEYISTLNYLYDRITNELTNSES |
| | | KNSAANVNYIAETLGINPAEFAEQYFRFSIMKEQKNLGFNITKLREVM |
| | | LDRKDMSEIRKNHKVFDSIRTKVYTMMDFVIYRYYIEEDAKVAAANKS |
| | | LPDNEKSLSEKDIFVINLRGSENDDQKDALYYDEANRIWRKLENIMHN |
| | | IKEFRGNKTREYKKKDAPRLPRILPAGRDVSAFSKLMYALTMELDGKE |
| | | INDLLTTLINKEDNIQSFLKVMPLIGVNAKFVEEYAFFKDSAKIADEL |
| | | RLIKSFARMGEPIADARRAMYIDAIRILGTNLSYDELKALADTESLDE |
| | | NGNKLKKGKHGMRNFIINNVISNKRFHYLIRYGDPAHLHEIAKNEAVV |
| | | KFVLGRIADIQKKQGONGKNQIDRYYETCIGKDKGKSVSEKVDALTKI |
| | | ITGMNYDQFDKKRSVIEDTGRENAEREKFKKIISLYLTVIYHILKNIV |
| | | NINARYVIGFHCVERDAQLYKEKGYDINLKKLEEKGFSSVTKLCAGID |
| | | ETAPDKRKDVEKEMAERAKESIDSLESANPKLYANYIKYSDEKKAEEF |
| | | TRQINREKAKTALNAYLRNTKWNVIIREDLLRIDNKTCTLFANKAVAL |
| | | EVARYVHAYINDIAEVNSYFQLYHYIMQRIIMNERYEKSSGKVSEYED |
| | | AVNDEKKYNDRLLKLLCVPFGYCIPRFKNLSIEALFDRNEAAKEDKEK |
| | | KKVSGNS |
| 3 | UrCas13d | MAKKNKMKPRELREAQKKAROLKAAEINNNAAPAIAAMPAAEVIAPAA |
| | | EKKKSSVKAAGMKSILVSENKMYITSFGKGNSAVLEYEVDNNDYNQTQ |
| | | LSSKDNSNIQLGGVNEVNITFSSKHGFESGVEINTSNPTHRSGESSPV |
| | | RGDMLGLKSELEKRFFGKTFDDNIHIQLIYNILDIEKILAVYVINIVY |
| | | ALNNMLGVKGSESHDDFIGYLSTNNIYDVFIDPDNSSLSDDKKANVRK |
| | | SLSKFNALLKTKRLGYFGLEEPKTKDNRVSQAYKKRVYHMLAIVGQIA |
| | | QCVFADKSGAKRFDLYSFINNIDPEYRDTLDYLVEERLKSINKDFIED |
| | | NKVNISLLIDMMKGYEADDIIRLYYDFIVLKSQKNLGESIKKLREKML |
| | | DEYGFRFKDKQYDSVRSKMYKLMDFLLFCNYYRNDIAAGESLVRKLRF |
| | | SMTDDEKEGIYADEAAKLWGKFRNDFENIADHMNGDVIKELGKADMDF |
| | | DEKILDSEKKNASDLLYFSKMIYMLTYFLDGKEINDLLTTLISKEDNI |
| | | KEFLKIMKSSAVDVECELTAGYKLENDSQRITNELFIVKNIASMRKPA |
| | | ASAKLTMFRDALTILGIDDKITDDRISGILKLKEKGKGIHGLRNFITN |
| | | NVIESSRFVYLIKYANAQKIREVAKNEKVVMFVLGGIPDTQIERYYKS |
| | | CVEFPDMNSSLGVKRSELARMIKNISFDDFKNVKQQAKGRENVAKERA |
| | | KAVIGLYLTVMYLLVKNLVNVNARYVIAIHCLERDFGLYKEIIPELAS |
| | | KNLKNDYRILSQTLCELCDKSPNLFLKKNERLRKCVEVDINNADSSMT |
| | | RKYANCIAALTVVRELKEYIGDICTVDSYFSIYHYVMQRCITKRENDT |
| | | KQEEKIKYEDDLLKNHGYTKDFVKALNSPFGYNIPRFKNLSIEQLEDR |
| | | NEYLTEK |
| 4 | RffCas13d | MKKKMSLREKREAEKQAKKAAYSAASKNTDSKPAEKKAETPKPAEIIS |
| | | DNSRNKTAVKAAGLKSTIISGDKLYMTSFGKGNAAVIEQKIDINDYSF |
| | | SAMKDTPSLEVDKAESKEISFSSHHPFVKNDKLTTYNPLYGGKDNPEK |
| | | PVGRDMLGLKDKLEERYFGCTENDNLHIQIIYNILDIEKILAVHSANI |
| | | TTALDHMVDEDDEKYLNSDYIGYMNTINTYDVEMDPSKNSSLSPKDRK |
| | | NIDNSRAKFEKLLSTKRLGYFGFDYDANGKDKKKNEEIKKRLYHLTAF |
| | | AGQLROWSFHSAGNYPRTWLYKLDSLDKEYLDTLDHYFDKRENDINDD |
| | | FVTKNATNLYILKEVFPEANFKDIADLYYDFIVIKSHKNMGESIKKLR |
| | | EKMLECDGADRIKEQDMDSVRSKLYKLIDFCIFKYYHEFPELSEKNVD |
| | | ILRAAVSDTKKDNLYSDEAARLWSIFKEKFLGFCDKIVVWVTGEHEKD |
| | | ITSVIDKDAYRNRSNVSYFSKLMYAMCFFLDGKEINDLLTTLINKEDN |
| | | IANQIKTAKELGINTAFVKNYDFFNHSEKYVDELNIVKNIARMKKPSS |
| | | NAKKAMYHDALTILGIPEDMDEKALDEELDLILEKKTDPVTGKPLKGK |
| | | NPLRNFIANNVIENSRFIYLIKFCNPENVRKIVNNTKVTEFVLKRIPD |
| | | AQIERYYKSCTDSEMNPPTEKKITELAGKLKDMNFGNFRNVRQSAKEN |
| | | MEKERFKAVIGLYLTVVYRVVKNLVDVNSRYIMAFHSLERDSQLYNVS |
| | | VDNDYLALTDTLVKEGDNSRSRYLAGNKRLRDCVKQDIDNAKKWFVSD |
| | | KYNSITKYRNNVAHLTAVRNCAEFIGDITKIDSYFALYHYLIQROLAK |
| | | GLDHERSGFDRNYPQYAPLFKWHTYVKDVVKALNAPFGYNIPRFKNLS |
| | | IDALFDRNEIKKNDGEKKSDD |
| 5 | RaCas13d | MAKKSKGMSLREKRELEKQKRIQKAAVNSVNDTPEKTEEANVVSVNVR |
| | | TSAENKHSKKSAAKALGLKSGLVIGDELYLTSFGRGNEAKLEKKISGD |
| | | TVEKLGIGAFEVAERDESTLTLESGRIKDKTARPKDPRHITVDTQGKF |
| | | KEDMLGIRSVLEKKIFGKTFDDNIHVOLAYNILDVEKIMAQYVSDIVY |
| | | MLHNTDKTERNDNLMGYMSIRNTYKTFCDTSNLPDDTKQKVENQKREF |
| | | DKIIKSGRLGYFGEAFMVNSGNSTKLRPEKEIYHIFALMASLROSYFH |
| | | GYVKDTDYQGTTWAYTLEDKLKGPSHEFRETIDKIFDEGFSKISKDFG |
| | | KMNKVNLQILEQMIGELYGSIERQNLTCDYYDFIQLKKHKYLGESIKR |
| | | LRETMLETTPAECYKAECYNSERQKLYKLIDFLIYDLYYNRKPARIEE |
| | | IVDKLRESVNDEEKESIYSVEAKYVYESLSKVLDKSLKNSVSGETIKD |
| | | LOKRYDDETANRIWDISQHSISGNVNCFCKLIYIMTLMLDGKEINDLL |
| | | TTLVNKFDNIASFIDVMDELGLEHSFTDNYKMFADSKAICLDLQFINS |
| | | FARMSKIDDEKSKRQLERDALVILDIGNKDETWINNYLDSDIFKLDKE |
| | | GNKLKGARHDFRNFIANNVIKSSRFKYLVKYSSADGMIKLKTNEKLIG |
| | | FVLDKLPETQIDRYYESCGLDNAVVDKKVRIEKLSGLIRDMKFDDESG |
| | | VKTSNKAGDNDKQDKAKYQAIISLYLMVLYQIVKNMIYVNSRYVIAFH |
| | | CLERDFGMYGKDFGKYYQGCRKLTDHFIEEKYMKEGKLGCNKKVGRYL |
| | | KNNISCCTDGLINTYRNQVDHFAVVRKIGNYAAYIKSIGSWFELYHYV |
| | | IQRIVFDEYRFALNNTESNYKNSIIKHHTYCKDMVKALNTPFGYDLPR |
| | | YKNLSIGDLFDRNNYLNKTKESIDANSSIDSQ |
| 6 | AdmCas13d | MNNKRKTKAKAAGLKSVFFDQKQAVLTTFAKGNNSQIEKKVVNSEVKD |
| | | LRQPPAFDLELKEKTFYISGKNNINTSRENPLASASLPLSKRQRIRAE |
| | | RIKRAREENRPYHNVKRVGEDDLRAKADLEKHYFGKEYSDNLKIQIIY |
| | | NILDINKIISPYINDIVYSMNNLARNDEYIDGKIDVIGSLSSTTDYSS |
| | | FMSPNKDLEKEKKFSFHRENYKKFVEASKPYMRYYGKVFIRDVKKSKL |
| | | STGKGEKIEVMYRSDEEIFTIFQILSYVRQSIMHNDIGNKSSILAIEK |
| | | YPARFVGFLSDLLKTKTNDVNRMFIDNNSQTNFWVLFSIFGLQDHTSG |
| | | ADKICRNFYDFVIKADSKNLGFSLKKIRELMLDLPNANMLRDHQFDTV |
| | | RSKFYTLLDFIIYQHYLEEKSRIDNMVEKLRMTLKEEEKEVLYAAEAK |
| | | IVWNAIGAKVINKLVPMMNGDALKEIKRKNRDRKLPQSVIATVQVNSD |
| | | ANVFSGLIYFLTLFLDGKEINEMVSNLITKFENIDSLLHVDREIYKSD |
| | | EKDLDLEIEKLALFFKGVVRPNAKTDTGAGEISKSFSIFOSAERIIEE |
| | | LKFIKNVTRMDNEIFPSEGVFLDAANVLGVRGDDFDFSNEFVGDDLHS |
| | | DANKKIINKINGTKEDRNLRNFIINNVVKSRRFQYIARHMNTHYVKQL |
| | | ANNETLNRFVLNKMGDAKIINRYYESISGNTPNIEVRSQIDYLVKRLR |
| | | SFSFEDLNDVKQKVRPGTNESIEKEKKKALVGLCLTIQYLVYKNLVNI |
| | | NARYTTAFYCLERDSKLKGFGVDVWRDFESYTALTNHFIKEGYLPVRK |
| | | AEILRANLKHLDCEDGFKYYRNQVTHLNAIRVAYKYINEIKSVHSYFA |
| | | LYHYIMQRHLYDSLQAKAKDSSGFVIDALKKSFEHKIYSKDLLHVLHS |
| | | PFGYNTARYKNLSIEALFDKNESRPEVNPLSTND |
| 7 | P1E0Cas13d | MEREVKKPPKKSLAKAAGLKSTFVISPQEKELAMTAFGRGNDALLQKR |
| | | IVDGVVRDVAGEKQQFQVORQDESRFRLONSRLADRTVTADDPLHRAE |
| | | TPRRQPLGAGMDQLRRKAILEQKYFGRTFDDNIHIQLIYNILDIHKML |
| | | AVPANHIVHTLNLLGGYGETDFVGMLPAGLPYDKLRVVKKKNGDTVDI |
| | | KADIAAYAKRPQLAYLGAAFYDVTPGKSKRDAARGRVKREQDVYAILS |
| | | LMSLLRQFCAHDSVRIWGQNTTAALYHLQALPQDMKDLLDDGWRRALG |
| | | GVNDHFLDTNKVNLLTLFEYYGAETKQARVALTQDFYRFVVLKEQKNM |
| | | GFSLRRLREELLKLPDAAYLTGQEYDSVRQKLYMLLDFLLCRLYAQER |
| | | ADRCEELVSALRCALSDEEKDTVYQAEAAALWQALGDTLRRKLLPLLK |
| | | GKKLQDKDKKKSDELGLSRDVLDGVLFRPAQQGSRANADYFCRLMHLS |
| | | TWFMDGKEINTLLTTLISKLENIDSLRSVLESMGLAYSFVPAYAMFDH |
| | | SRYIAGQLRVVNNIARMRKPAIGAKREMYRAAVVLLGVDSPEAAAAIT |
| | | DDLLQIDPETGKVRPRSDSARDTGLRNFIANNVVESRRFTYLLRYMTP |
| | | EQARVLAQNEKLIAFVLSTVPDTQLERYCRTCGREDITGRPAQIRYLT |
| | | AQIMGVRYESFTDVEQRGRGDNPKKERYKALIGLYLTVLYLAVKNMVN |
| | | CNARYVIAFYCRDRDTALYQKEVCWYDLEEDKKSGKQRQVEDYTALTR |
| | | YFVSQGYLNRHACGYLRSNMNGISNSLLTAYRNAVDHLNAIPPLGSLC |
| | | RDIGRVDSYFALYHYAVQQYLNGRYYRKTPREQELFAAMAQHRTWCSD |
| | | LVKALNTPFGYNLARYKNLSIDGLEDREGDHVVREDGEKPAE |
| 8 | EsCas13d | MGKKIHARDLREQRKTDRTEKFADQNKKREAERAVPKKDAAVSVKSVS |
| | | SVSSKKDNVTKSMAKAAGVKSVFAVGNTVYMTSFGRGNDAVLEQKIVD |
| | | TSHEPLNIDDPAYQLNVVTMNGYSVTGHRGETVSAVTDNPLRRENGRK |
| | | KDEPEQSVPTDMLCLKPTLEKKFFGKEFDDNIHIQLIYNILDIEKILA |
| | | VYSTNAIYALNNMSADENIENSDFFMKRTTDETFDDFEKKKESTNSRE |
| | | KADFDAFEKFIGNYRLAYFADAFYVNKKNPKGKAKNVLREDKELYSVL |
| | | TLIGKLRHWCVHSEEGRAEFWLYKLDELKDDFKNVLDVVYNRPVEEIN |
| | | NRFIENNKVNIQILGSVYKNTDIAELVRSYYEFLITKKYKNMGFSIKK |
| | | LRESMLEGKGYADKEYDSVRNKLYQMTDFILYTGYINEDSDRADDLVN |
| | | TLRSSLKEDDKTTVYCKEADYLWKKYRESIREVADALDGDNIKKLSKS |
| | | NIEIQEDKLRKCFISYADSVSEFTKLIYLLTRFLSGKEINDLVTTLIN |
| | | KEDNIRSFLEIMDELGLDRTFTAEYSFFEGSTKYLAELVELNSFVKSC |
| | | SFDINAKRTMYRDALDILGIESDKTEEDIEKMIDNILQIDANGDKKLK |
| | | KNNGLRNFIASNVIDSNRFKYLVRYGNPKKIRETAKCKPAVRFVLNEI |
| | | PDAQIERYYEACCPKNTALCSANKRREKLADMIAEIKFENFSDAGNYQ |
| | | KANVTSRTSEAEIKRKNQAIIRLYLTVMYIMLKNLVNVNARYVIAFHC |
| | | VERDTKLYAESGLEVGNIEKNKTNLTMAVMGVKLENGIIKTEFDKSFA |
| | | ENAANRYLRNARWYKLILDNLKKSERAVVNEFRNTVCHLNAIRNININ |
| | | IKEIKEVENYFALYHYLIQKHLENRFADKKVERDTGDFISKLEEHKTY |
| | | CKDFVKAYCTPFGYNLVRYKNLTIDGLEDKNYPGKDDSDEQK |
| 9 | PspCas13b | MNIPALVENQKKYFGTYSVMAMLNAQTVLDHIQKVADIEGEQNENNEN |
| | | LWFHPVMSHLYNAKNGYDKQPEKTMFIIERLQSYFPFLKIMAENQREY |
| | | SNGKYKQNRVEVNSNDIFEVLKRAFGVLKMYRDLTNHYKTYEEKLNDG |
| | | CEFLTSTEQPLSGMINNYYTVALRNMNERYGYKTEDLAFIQDKRFKFV |
| | | KDAYGKKKSQVNTGFFLSLQDYNGDTQKKLHLSGVGIALLICLFLDKQ |
| | | YINIFLSRLPIFSSYNAQSEERRIIIRSFGINSIKLPKDRIHSEKSNK |
| | | SVAMDMLNEVKRCPDELFTTLSAEKQSRFRIISDDHNEVLMKRSSDRF |
| | | VPLLLQYIDYGKLFDHIRFHVNMGKLRYLLKADKTCIDGQTRVRVIEQ |
| | | PLNGFGRLEEAETMRKQENGTFGNSGIRIRDFENMKRDDANPANYPYI |
| | | VDTYTHYILENNKVEMFINDKEDSAPLLPVIEDDRYVVKTIPSCRMST |
| | | LEIPAMAFHMFLFGSKKTEKLIVDVHNRYKRLFQAMQKEEVTAENIAS |
| | | FGIAESDLPQKILDLISGNAHGKDVDAFIRLTVDDMLTDTERRIKRFK |
| | | DDRKSIRSADNKMGKRGFKQISTGKLADFLAKDIVLFQPSVNDGENKI |
| | | TGLNYRIMQSAIAVYDSGDDYEAKQQFKLMFEKARLIGKGTTEPHPFL |
| | | YKVFARSIPANAVEFYERYLIERKFYLTGLSNEIKKGNRVDVPFIRRD |
| | | QNKWKTPAMKTLGRIYSEDLPVELPRQMFDNEIKSHLKSLPQMEGIDE |
| | | NNANVTYLIAEYMKRVLDDDFQTFYQWNRNYRYMDMLKGEYDRKGSLQ |
| | | HCFTSVEEREGLWKERASRTERYRKQASNKIRSNRQMRNASSEEIETI |
| | | LDKRLSNSRNEYQKSEKVIRRYRVQDALLFLLAKKTLTELADFDGERF |
| | | KLKEIMPDAEKGILSEIMPMSFTFEKGGKKYTITSEGMKLKNYGDFFV |
| | | LASDKRIGNLLELVGSDIVSKEDIMEEFNKYDQCRPEISSIVENLEKW |
| | | AFDTYPELSARVDREEKVDEKSILKILLNNKNINKEQSDILRKIRNAF |
| | | DHNNYPDKGVVEIKALPEIAMSIKKAFGEYAIMKGSLQ |
| 10 | PguCas13b | MTEQSERPYNGTYYTLEDKHFWAAFLNLARHNAYITLTHIDROLAYSK |
| | | ADITNDQDVLSFKALWKNFDNDLERKSRLRSLILKHFSFLEGAAYGKK |
| | | LFESKSSGNKSSKNKELTKKEKEELQANALSLDNLKSILFDFLQKLKD |
| | | FRNYYSHYRHSGSSELPLFDGNMLQRLYNVFDVSVQRVKIDHEHNDEV |
| | | DPHYHFNHLVRKGKKDRYGHNDNPSFKHHFVDGEGMVTEAGLLFFVSL |
| | | FLEKRDAIWMQKKIRGFKGGTETYQQMTNEVFCRSRISLPKLKLESLR |
| | | MDDWMLLDMLNELVRCPKPLYDRLREDDRACFRVPVDILPDEDDTDGG |
| | | GEDPFKNTLVRHQDRFPYFALRYFDLKKVFTSLRFHIDLGTYHFAIYK |
| | | KMIGEQPEDRHLTRNLYGFGRIQDFAEEHRPEEWKRLVRDLDYFETGD |
| | | KPYISQTSPHYHIEKGKIGLRFMPEGQHLWPSPEVGTTRTGRSKYAQD |
| | | KRLTAEAFLSVHELMPMMFYYFLLREKYSEEVSAERVQGRIKRVIEDV |
| | | YAVYDAFARDEINTRDELDACLADKGIRRGHLPRQMIAILSQEHKDME |
| | | EKIRKKLQEMMADTDHRLDMLDRQTDRKIRIGRKNAGLPKSGVIADWL |
| | | VRDMMRFQPVAKDASGKPLNNSKANSTEYRMLQRALALEGGEKERLTP |
| | | YFROMNLTGGNNPHPFLHETRWESHTNILSFYRSYLRARKAFLERIGR |
| | | SDRVENRPFLLLKEPKTDRQTLVAGWKGEFHLPRGIFTEAVRDCLIEM |
| | | GHDEVASYKEVGFMAKAVPLYFERACEDRVOPFYDSPFNVGNSLKPKK |
| | | GRFLSKEERAEEWERGKERFRDLEAWSYSAARRIEDAFAGIEYASPGN |
| | | KKKIEQLLRDLSLWEAFESKLKVRADRINLAKLKKEILEAQEHPYHDF |
| | | KSWQKFERELRLVKNQDIITWMMCRDLMEENKVEGLDTGTLYLKDIRP |
| | | NVQEQGSLNVLNRVKPMRLPVVVYRADSRGHVHKEEAPLATVYIEERD |
| | | TKLLKQGNFKSFVKDRRLNGLFSFVDTGGLAMEQYPISKLRVEYELAK |
| | | YQTARVCVFELTLRLEESLLTRYPHLPDESFREMLESWSDPLLAKWPE |
| | | LHGKVRLLIAVRNAFSHNQYPMYDEAVESSIRKYDPSSPDAIEERMGL |
| | | NIAHRLSEEVKQAKETVERIIQAGSLQ |
| 11 | RanCas13b | MEKPLLPNVYTLKHKFFWGAFLNIARHNAFITICHINEQLGLKTPSND |
| | | DKIVDVVCETWNNILNNDHDLLKKSQLTELILKHFPFLTAMCYHPPKK |
| | | EGKKKGHQKEQQKEKESEAQSQAEALNPSKLIEALEILVNQLHSLRNY |
| | | YSHYKHKKPDAEKDIFKHLYKAFDASLRMVKEDYKAHFTVNLTRDFAH |
| | | LNRKGKNKQDNPDFNRYRFEKDGFFTESGLLFFTNLFLDKRDAYWMLK |
| | | KVSGFKASHKOREKMTTEVFCRSRILLPKLRLESRYDHNQMLLDMLSE |
| | | LSRCPKLLYEKLSEENKKHFQVEADGFLDEIEEEQNPFKDTLIRHQDR |
| | | FPYFALRYLDLNESFKSIRFQVDLGTYHYCIYDKKIGDEQEKRHLTRT |
| | | LLSFGRLQDFTEINRPQEWKALTKDLDYKETSNQPFISKTTPHYHITD |
| | | NKIGFRLGTSKELYPSLEIKDGANRIAKYPYNSGFVAHAFISVHELLP |
| | | LMFYQHLTGKSEDLLKETVRHIQRIYKDFEEERINTIEDLEKANQGRL |
| | | PLGAFPKQMLGLLQNKQPDLSEKAKIKIEKLIAETKLLSHRLNTKLKS |
| | | SPKLGKRREKLIKTGVLADWLVKDEMRFQPVAYDAQNQPIKSSKANST |
| | | EFWFIRRALALYGGEKNRLEGYFKQTNLIGNTNPHPFLNKENWKACRN |
| | | LVDFYQQYLEQREKFLEAIKNOPWEPYQYCLLLKIPKENRKNLVKGWE |
| | | QGGISLPRGLFTEAIRETLSEDLMLSKPIRKEIKKHGRVGFISRAITL |
| | | YFKEKYQDKHQSFYNLSYKLEAKAPLLKREEHYEYWQQNKPQSPTESQ |
| | | RLELHTSDRWKDYLLYKRWQHLEKKLRLYRNQDVMLWLMTLELTKNHF |
| | | KELNLNYHQLKLENLAVNVQEADAKLNPLNQTLPMVLPVKVYPATAFG |
| | | EVQYHKTPIRTVYIREEHTKALKMGNFKALVKDRRLNGLFSFIKEEND |
| | | TQKHPISQLRLRRELEIYQSLRVDAFKETLSLEEKLLNKHTSLSSLEN |
| | | EFRALLEEWKKEYAASSMVTDEHIAFIASVRNAFCHNQYPFYKEALHA |
| | | PIPLFTVAQPTTEEKDGLGIAEALLKVLREYCEIVKSQI |
| 12 | eIF4A3 | MATTATMATSGSARKRLLKEEDMTKVEFETSEEVDVTPTFDTMGLRED |
| | | LLRGIYAYGFEKPSAIQQRAIKQIIKGRDVIAQSQSGTGKTATFSISV |
| | | LOCLDIQVRETQALILAPTRELAVOIQKGLLALGDYMNVQCHACIGGT |
| | | NVGEDIRKLDYGQHVVAGTPGRVFDMIRRRSLRTRAIKMLVLDEADEM |
| | | LNKGFKEQIYDVYRYLPPATQVVLISATLPHEILEMTNKEMTDPIRIL |
| | | VKRDELTLEGIKOFFVAVEREEWKFDTLCDLYDTLTITQAVIFCNTKR |
| | | KVDWLTEKMREANFTVSSMHGDMPQKERESIMKEFRSGASRVLISTDV |
| | | WARGLDVPQVSLIINYDLPNNRELYIHRIGRSGRYGRKGVAINFVKND |
| | | DIRILRDIEQYYSTQIDEMPMNVADLI |
| 13 | Y14/RBM8A | MADVLDLHEAGGEDFAMDEDGDESIHKLKEKAKKRKGRGFGSEEGSRA |
| | (minimal | RMREDYDSVEQDGDEPGPQRSVEGWILFVTGVHEEATEEDIHDKFAEY |
| | sequence for | GEIKNIHLNLDRRTGYLKGYTLVEYETYKEAQAAMEGLNGQDLMGQPI |
| | NMD | SVDWCFVRGPPKGKRRGGRRRSRSPDRRRR |
| | activation | |
| | is | |
| | underlined). | |
| 14 | MAGOH | MESDFYLRYYVGHKGKFGHEFLEFEFRPDGKLRYANNSNYKNDVMIRK |
| | | EAYVHKSVMEELKRIIDDSEITKEDDALWPPPDRVGRQELEIVIGDEH |
| | | ISFTTSKIGSLIDVNQSKDPEGLRVFYYLVQDLKCLVFSLIGLHFKIK |
| | | PI |
| 15 | UPF1 | MSVEAYGPSSQTLTFLDTEEAELLGADTQGSEFEFTDFTLPSQTQTPP |
| | | GGPGGPGGGGAGGPGGAGAGAAAGOLDAQVGPEGILONGAVDDSVAKT |
| | | SQLLAELNFEEDEEDTYYTKDLPIHACSYCGIHDPACVVYCNTSKKWF |
| | | CNGRGNTSGSHIVNHLVRAKCKEVTLHKDGPLGETVLECYNCGCRNVE |
| | | LLGFIPAKADSVVVLLCROPCASOSSLKDINWDSSQWQPLIQDRCFLS |
| | | WLVKIPSEQEQLRARQITAQQINKLEELWKENPSATLEDLEKPGVDEE |
| | | PQHVLLRYEDAYQYQNIFGPLVKLEADYDKKLKESQTQDNITVRWDLG |
| | | LNKKRIAYFTLPKTDSDMRLMQGDEICLRYKGDLAPLWKGIGHVIKVP |
| | | DNYGDEIAIELRSSVGAPVEVTHNFQVDFVWKSTSFDRMQSALKTFAV |
| | | DETSVSGYIYHKLLGHEVEDVIIKCOLPKRFTAQGLPDLNHSQVYAVK |
| | | TVLQRPLSLIQGPPGTGKTVTSATIVYHLARQGNGPVLVCAPSNIAVD |
| | | QLTEKIHQTGLKVVRLCAKSREAIDSPVSFLALHNQIRNMDSMPELOK |
| | | LQQLKDETGELSSADEKRYRALKRTAERELLMNADVICCTCVGAGDPR |
| | | LAKMQFRSILIDESTQATEPECMVPVVLGAKQLILVGDHCQLGPVVMC |
| | | KKAAKAGLSQSLFERLVVLGIRPIRLQVQYRMHPALSAFPSNIFYEGS |
| | | LQNGVTAADRVKKGFDFQWPQPDKPMFFYVTQGQEEIASSGTSYLNRT |
| | | EAANVEKITTKLLKAGAKPDQIGIITPYEGORSYLVQYMQFSGSLHTK |
| | | LYQEVEIASVDAFQGREKDFIILSCVRANEHQGIGFLNDPRRLNVALT |
| | | RARYGVIIVGNPKALSKQPLWNHLLNYYKEQKVLVEGPLNNLRESLMQ |
| | | FSKPRKLVNTINPGARFMTTAMYDAREAIIPGSVYDRSSQGRPSSMYF |
| | | QTHDQIGMISAGPSHVAAMNIPIPFNLVMPPMPPPGYFGQANGPAAGR |
| | | GTPKGKTGRGGRQKNRFGLPGPSQTNLPNSQASQDVASQPFSQGALTQ |
| | | GYISMSQPSQMSQPGLSQPELSQDSYLGDEFKSQIDVALSQDSTYQGE |
| | | RAYQHGGVTGLSQY |
| 16 | UPF2 | MDHPEDESHSEKQDDEEALARLEEIKKSIEAKLTLRQNNLNPERPDSA |
| | (minimal | YLRTLDSSIKRNTAVIKKLKQINEEQREGLMDDLRGVNLSKFVSEAVT |
| | sequence for | AICEAKLKSSDIQAAVQICSLLHQRYKEFSASLTQGLLKVFFPGKSAE |
| | NMD | DLEADKNSKAMKKRSTLKLLLELYYVGVIEDSNIFINIIKDLTSVEQL |
| | activation | KDRDTTQTNLTLLTSFARQGRIFLGLPISGQDEDFFKGLDVTADQKKS |
| | is | FKKAFNTYYDALADLLQSEHKLLLQMEKENAKLVNAKGELSEDSASSY |
| | underlined) | EKLRKSYDHLYRNISSLAEALDMQPPVMPEDGTTRLTAGDEASPSGTV |
| | | KDTSVPEPIWDDEDTKTFYECLPDLRAFVPAVLLGEAEPKSNEQSAKA |
| | | KEKLSESSSEVVENQQTTEDTTEVSADSASMDDRSNAEQPKEKEEVEK |
| | | EKAKDTKKEKGKEKDSEKKMEHEKEKGKSLDVANFERLLORLPGCVSR |
| | | DLIDQLTVEYCYLNSKTNRKKLVKALFNVPRTSLELLAYYSRMVATLA |
| | | SCMKDIPSMLVQMLEDEFNSLVHKKDQMNIETKIRNIRFIGELCKFKI |
| | | VPAGLVFSCLKACLDEFTHHNIDVACNLLETCGRFLYRSPETTLRMTN |
| | | MLDILMRLKNVKNLDPROSTLVENAYYLCKPPERSARISKVRPPLHQY |
| | | VRKLLFSDLDKDSIANVLKQLRKLPWSECEQYILKCFMKVHKGKYGQI |
| | | HLIASLTSGLSRHHDEFVVAVVDEVLEEIRVGLELNEYGAQQKRLAHM |
| | | RFLGELYNYEHVDSSVIFETLYLTLLYGHDTSEQEVLDPPEDFFRVRM |
| | | VIILLETCGHYFDRGSSKKRLDQFLIHFQRYILSKGHLPLDIEFDLQD |
| | | LFANLRPNMTRYSTIDEVNAAILQLEEREHASSGDKVSIERHSDTKPS |
| | | NKSSSDVISSNGKSTAKDIRENGEAHGEESDSDSGSGSVVRDGONEEL |
| | | DDGNHERGSESGDGDDYDDGDGPGSDDDKFRVRQKVVTVDLEEQADED |
| | | QELKALLQESMEQRKLELRGRPALNMTIPMSVFEGSGKDHHHFGRVVG |
| | | ENGEEVLDEENGEQREVQVKVLVKRGNKQQTROMLIPSDCALVOSTKQ |
| | | KEAAELEEKQDIKRLVLEYNERDEEEANGLGTQILNWTSGGSRGSTRT |
| | | GEGSGKSGGSRHRFYYHQGGGGSYHARRK |
| 17 | UPF3B/3X | MKEEKEHRPKEKRVTLLTPAGATGSGGGTSGDSSKGEDKQDRNKEKKE |
| | | ALSKVVIRRLPPTLTKEQLQEHLQPMPEHDYFEFFSNDTSLYPHMYAR |
| | | AYINFKNQEDIILFRDRFDGYVFLDNKGQEYPAIVEFAPFQKAAKKKT |
| | | KKRDTKVGTIDDDPEYRKFLESYATDNEKMTSTPETLLEEIEAKNREL |
| | | IAKKTTPLLSFLKNKORMREEKREERRRREIERKRQREEERRKWKEEE |
| | | KRKRKDIEKLKKIDRIPERDKLKDEPKIKVHRFLLQAVNOKNLLKKPE |
| | | KGDEKELDKREKAKKLDKENLSDERASGOSCTLPKRSDSELKDEKPKR |
| | | PEDESGRDYREREREYERDQERILRERERLKRQEEERRRQKERYEKEK |
| | | TFKRKEEEMKKEKDTLRDKGKKAESTESIGSSEKTEKKEEVVKRDRIR |
| | | NKDRPAMQLYQPGARSRNRLCPPDDSTKSGDSAAERKQESGISHRKEG |
| | | GEE |
| 18 | Nuclear | NVKKRKRRSKKINKLTDGQIRINHVSSEKKRRE |
| | Export | |
| | Signal | |
| 19 | Nuclear | LQLPPLERLTL |
| | Export | |
| | Signal | |
| 20 | Exemplary | CACUAGUGCGAAUUUGCACUAGUCUAAAAC |
| | hairpin | |
| | sequence of | |
| | guide RNA | |
| 21 | Exemplary | TGGTTCACTCTGAACGGAGCTGG |
| | guide RNA | |
| | sequence | |
| 22 | Exemplary | GS |
| | linker | |
| | sequence | |
| 23 | Exemplary | GSGGGGS |
| | linker | |
| | sequence | |
| 24 | Exemplary | GGGGSGGGGSGGGGS |
| | linker | |
| | sequence | |
| 25 | Exemplary | GGSGGSGGSGGSGGSGGS |
| | linker | |
| | sequence | |
| 26 | Exemplary | SGGSSGGSSGSETPGTSESATPESSGGSSGGS |
| | linker | |
| | sequence | |
| 27 | RNPS1 | MDLSGVKKKSLLGVKENNKKSSTRAPSPTKRKDRSDEKSKDRSKDKGA |
| | | TKESSEKDRGRDKTRKRRSASSGSSSTRSRSSSTSSSGSSTSTGSSSG |
| | | SSSSSASSRSGSSSTSRSSSSSSSSGSPSPSRRRHDNRRRSRSKSKPP |
| | | KRDEKERKRRSPSPKPTKVHIGRLTRNVTKDHIMEIFSTYGKIKMIDM |
| | | PVERMHPHLSKGYAYVEFENPDEAEKALKHMDGGQIDGQEITATAVLA |
| | | PWPRPPPRRFSPPRRMLPPPPMWRRSPPRMRRRSRSPRRRSPVRRRSR |
| | | SPGRRRHRSRSSSNSSR |
| 28 | SRSF1 | MSGGGVIRGPAGNNDCRIYVGNLPPDIRTKDIEDVFYKYGAIRDIDLK |
| | | NRRGGPPFAFVEFEDPRDAEDAVYGRDGYDYDGYRLRVEFPRSGRGTG |
| | | RGGGGGGGGGAPRGRYGPPSRRSENRVVVSGLPPSGSWQDLKDHMREA |
| | | GDVCYADVYRDGTGVVEFVRKEDMTYAVRKLDNTKFRSHEGETAYIRV |
| | | KVDGPRSPSYGRSRSRSRSRSRSRSRSNSRSRSYSPRRSRGSPRYSPR |
| | | HSRSRSRT |

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A fusion protein comprising:
a RNA binding domain;
an exon-junction complex (EJC) mimicking domain; and
at least one nuclear export signal,
wherein the RNA binding domain comprises a Cas nuclease or a RNA binding region thereof and wherein the EJC mimicking domain comprises one or more regions derived from one or more components of EJC.

2. (canceled)

3. The fusion protein of claim 1, wherein the nuclear export signal comprises SEQ ID NO: 18 or 19.

4. The fusion protein of claim 1, wherein the RNA binding domain comprises a catalytically inactive Cas nuclease or a RNA binding region thereof.

5. The fusion protein of claim 1, wherein the RNA binding domain comprises a catalytically inactive Cas13d nuclease or a RNA binding region thereof.

6. The fusion protein of claim 1, wherein the RNA binding domain comprises SEQ ID NO:2.

7. The fusion protein of claim 1, wherein the component of the EJC is selected from the group consisting of elF4A3, Y14/RBM8A, MAGOH, RNPS1, SRSF1, UPF1, UPF2 and UPF3B/3X.

8. The fusion protein of claim 7, wherein (1) the elF4A3 comprises SEQ ID NO: 12; (2) the Y14/RBM8A comprises SEQ ID NO: 13; (3) the MAGOH comprises SEQ ID NO: 14; (4) the UPF1 comprises SEQ ID NO: 15; (5) the UPF2 comprises SEQ ID NO: 16; (6) the UPF3B/3X comprises SEQ ID NO: 17; (7) the RNPS1 comprises SEQ ID NO: 27; and (8) the SRSF1 comprises SEQ ID NO: 28.

9. The fusion protein of claim 1, further comprising a linker between the RNA binding domain and the EJC mimicking domain.

10. An expression vector comprising:
a first nucleotide sequence encoding a RNA binding domain of a CAS nuclease;
a second nucleotide sequence encoding an exon-junction complex (EJC) mimicking domain derived from a component of EJC;
a third nucleotide sequence encoding a nuclear export signal; and
a regulatory sequence operably linked to at least one of the first, the second, and the third nucleotide sequences.

11. (canceled)

12. The expression vector of claim 10, wherein the first nucleotide sequence encodes dCas13d.

13. The expression vector of claim 10, wherein the second nucleotide sequence encodes one or more proteins or peptides or fragments thereof derived from elF4A3, Y14/RBM8A, MAGOH, RNPS1, SRSF1, UPF1, UPF2 or UPF3B/3X, wherein the amino acid sequence is capable of activation of NMD.

14. The expression vector of claim 10, further comprising a nucleotide sequence encoding a guide RNA.

15. The expression vector of claim 14, wherein the guide RNA comprises a sequence complementary to a target sequence in a NMD-insensitive PTC-containing mRNA, wherein the 5′ end of the target sequence is located at least 10 nucleotides downstream from the NMD-insensitive PTC in the mRNA.

16. The expression vector of claim 10, wherein the expression vector is a non-viral vector.

17. The expression vector of claim 10,
wherein the expression vector is a viral vector.

18. The expression vector of claim 17, wherein the expression vector is an AAV vector.

19. A pharmaceutical composition comprising:
the expression vector of claim 10; and
a pharmaceutically acceptable carrier.

20. An artificial exon-junction complex comprising:
a fusion protein comprising a RNA binding domain, an exon-junction complex (EJC) mimicking domain, and a nuclear export signal;
a guide RNA; and
a target mRNA
wherein the RNA binding domain comprises a RNA binding region of a CAS nuclease, wherein the (EJC) mimicking domain comprises one or more regions that are derived from a component of EJC,
wherein the guide RNA comprises a sequence complementary to a target sequence in the target mRNA,
wherein the target mRNA comprises a premature termination codon (PTC) that is insensitive to nonsense-mediated mRNA decay (NMD), and
wherein the 5′ end of the target sequence is located at least 10 nucleotides downstream from the PTC in the target mRNA.

21. (canceled)

22. A method for treating a disease or a condition in a subject caused by translation of a target mRNA comprising a premature termination codon (PTC) that is insensitive to the nonsense-mediated mRNA decay (NMD) process, the method comprises the steps of:
introducing into cells of the subject expressing the target mRNA:
(1) the fusion protein of claim 1, and
(2) a guide RNA that binds specifically to a target region in the target mRNA,
wherein the fusion protein and the guide RNA form a complex that binds to the target mRNA and results in the degradation of the target mRNA by NMD.

23-24. (canceled)

25. The method of claim 22, wherein the 5′-end of the target region is at least 10 nucleotides downstream of the PTC in the target mRNA.

26-28. (canceled)

29. A method for inducing mRNA decay of a target mRNA comprising a premature termination codon (PTC) that is insensitive to the nonsense-mediated mRNA decay (NMD) process, the method comprises the steps of:
introducing into cells expressing the target mRNA:
(a) the expression vector of claim 10 and
(b) a guide RNA that binds specifically to a target region in the target mRNA;
wherein the expression vector expresses a fusion protein comprising a RNA binding domain and the EJC mimicking domain in the cells.

30-32. (canceled)

33. The method of claim 29, wherein the guide RNA comprises a sequence complementary to a target sequence in a NMD-insensitive PTC-containing mRNA, wherein the 5′ end of the target sequence is located at least 10 nucleotides downstream from the NMD-insensitive PTC in the mRNA.

34. The method of claim 33, wherein the guide RNA is introduced into the cells in the form of a second expression vector comprising a nucleotide sequence encoding the guide RNA.

35. The method of claim 33, wherein the guide RNA is included in the same expression vector that expresses the fusion protein.
